(19) United States
(12) Patent Application Publication
(10) Pub. No.: US 2022/0298233 A1
(43) Pub. Date: Sep. 22, 2022

(54) Methods for Treatment of Subjects with Psoriatic Arthritis

(71) Applicant: Sun Pharmaceutical Industries Limited, Goregaon (East), Mumbai (IN)

(72) Inventors: Kirti Wardhaman Ganorkar, Goregaon (East), Mumbai (IN); Atul Mathuradas Raut, Powai, Mumbai (IN); Anil Raghavan, Vile Parle (West), Mumbai, Maharashtra (IN); Siu-Long Yao, Princeton, NJ (US)

(21) Appl. No.: 17/603,921

(22) PCT Filed: Apr. 15, 2020
(86) PCT No.: PCT/IB2020/053565

§ 371 Date: Oct. 14, 2021

(30) Foreign Application Priority Data
Apr. 15, 2019 (IN) 201921015050
Jan. 31, 2020 (IN) 202021004422

(51) Int. Cl. C07K 16/24; A61P 19/02

(52) U.S. Cl. CPC C07K 16/244; A61P 19/02; A61K 2039/505

(57) ABSTRACT

This disclosure relates to an anti-IL-23p19 antibody hum13B8-b or antigen binding fragment thereof and its use in the treatment of psoriatic arthritis.

### FIELD OF THE DISCLOSURE

This disclosure relates to an anti-IL-23p19 antibody hum13B8-b or antigen binding fragment thereof and its use in the treatment of psoriatic arthritis. In some embodiments, the disclosure relates to a method of treating psoriatic arthritis wherein the treatment results in improvement from the baseline value of both tender joint count and swollen joint count. In some embodiments, the disclosure relates to a method of determining the efficacy of an anti-IL-23p19 antibody for the treatment of psoriatic arthritis.

### BACKGROUND

Psoriasis (PsO) is a chronic inflammatory skin disorder affecting approximately 1% to 2% of people worldwide. Psoriatic arthritis (PsA) has been defined as a unique inflammatory arthritis associated with PsO. The precise prevalence is unknown, but estimates vary from 0.3% to 1% of the population; among patients with PsO, the observed prevalence of inflammatory arthritis varies from 6% to 42%. The clinical features typically present as an oligoarticular and mild disease. However, with time PsA becomes polyarticular, and it is a severe disease in at least 20% of patients. Gladman et al., Ann. Rheum. Dis. 64(Suppl II): ii14—ii17 (2005). Symptoms include tenderness, pain and stiffness in and around the joints, dactylitis, spondylitis, pain and swelling in the heels, nail disfiguration (discoloration, splitting, or pitting), and generalized fatigue. Patients with PsA who present with polyarticular disease are at risk for disease progression. In addition to progression of clinical and radiological damage, health related quality of life is reduced among patients with PsA. Gladman et al. (2005).

Current treatment choices for PsA include non-steroidal anti-inflammatory drugs (NSAIDs), corticosteroids, topical treatments (for skin), light therapy (for skin), physiotherapy, and disease-modifying anti-rheumatic drug (DMARDs). There are also two types of biologics approved for the use in treating PsA, and more recently an agent that targets interleukin-12 (IL-12) and IL-23. Methotrexate (MTX) is approved by the U.S. Food and Drug Administration (FDA) for the skin condition PsO, but it is frequently used off-label for PsA. Methotrexate has been reported as providing symptomatic relief to some patients with multiple joint involvement and PsO but there is little scientific evidence to support the use as a disease-modifying agent for PsA.

### SUMMARY

Provided herein is a method of treating psoriatic arthritis comprising administering an anti-IL-23p19 antibody hum13B8-b to a patient in need thereof, wherein the patient is subcutaneously administered a first dose of the antibody on week 0 and a subsequent dose at every 12 weeks thereafter; and wherein the antibody hum13B8-b comprises: (i) a light chain polypeptide comprising the amino acid sequence of SEQ ID NO: 1; and (ii) a heavy chain polypeptide comprising the amino acid sequence of SEQ ID NO: 2.

Also provided herein is a method of treating psoriatic arthritis comprising administering to a patient in need thereof a therapeutically effective amount of an anti-IL-23p19 antibody hum13B8-b, wherein the treatment results in at least 20% improvement from baseline value of tender joint count and swollen joint count; and wherein the antibody hum13B8-b comprises: (i) a light chain polypeptide comprising the amino acid sequence of SEQ ID NO: 1; and (ii) a heavy chain polypeptide comprising the amino acid sequence of SEQ ID NO: 2.

Further provided herein is a method of treating psoriatic arthritis comprising administering to a patient in need thereof a therapeutically effective amount of an anti-IL-23p19 antibody hum13B8-b, wherein the treatment results in at least 50% improvement from baseline value of tender joint count and swollen joint count; and wherein the antibody hum13B8-b comprises: (i) a light chain polypeptide comprising the amino acid sequence of SEQ ID NO: 1; and (ii) a heavy chain polypeptide comprising the amino acid sequence of SEQ ID NO: 2.

Further provided herein is a method of treating psoriatic arthritis comprising administering to a patient in need thereof a therapeutically effective amount of an anti-IL-23p19 antibody hum13B8-b, wherein the treatment results in at least 70% improvement from baseline value of tender joint count and swollen joint count; and wherein the antibody hum13B8-b comprises: (i) a light chain polypeptide comprising the amino acid sequence of SEQ ID NO: 1; and (ii) a heavy chain polypeptide comprising the amino acid sequence of SEQ ID NO: 2.

Further provided herein is a method of determining the efficacy of an anti-IL-23p19 antibody for the treatment of psoriatic arthritis comprising administering an anti-IL-23p19 antibody hum 13B8-b to a patient, wherein the patient is subcutaneously administered a first dose of the antibody on week 0 and a subsequent dose at every 12 weeks after the first dose; and wherein the antibody hum13B8-b comprises: (i) a light chain polypeptide comprising the amino acid sequence of SEQ ID NO: 1; and (ii) a heavy chain polypeptide comprising the amino acid sequence of SEQ ID NO: 2, and wherein an ACR20 response value of at least about 40% at week 24 or week 52 indicates the efficacy of the antibody.

Further provided herein is a method of determining the efficacy of an anti-IL-23p19 antibody for the treatment of psoriatic arthritis comprising administering an anti-IL-23p19 antibody hum 13B8-b to a patient, wherein the patient is subcutaneously administered a first dose of the antibody on week 0 and a subsequent dose at every 12 weeks after the first dose; and wherein the antibody hum13B8-b comprises: (i) a light chain polypeptide comprising the amino acid sequence of SEQ ID NO: 1; and (ii) a heavy chain polypeptide comprising the amino acid sequence of SEQ ID NO: 2, and wherein an ACR50 response value of at least about 20% at week 24 or week 52 indicates the efficacy of the antibody.

Further provided herein is a method of determining the efficacy of an anti-IL-23p19 antibody for the treatment of psoriatic arthritis comprising administering an anti-IL-23p19 antibody hum 13B8-b to a patient, wherein the patient is subcutaneously administered a first dose of the antibody on week 0 and a subsequent dose at every 12 weeks after the first dose; and wherein the antibody hum13B8-b comprises: (i) a light chain polypeptide comprising the amino acid sequence of SEQ ID NO: 1; and (ii) a heavy chain polypeptide comprising the amino acid sequence of SEQ ID NO: 2, and wherein an ACR70 response value of at least 10% at week 24 or week 52 indicates the efficacy of the antibody.

In some embodiments provided herein is a method of determining the efficacy of an anti-IL-23p19 antibody for the treatment of psoriatic arthritis comprising administering an anti-IL-23p19 antibody hum13B8-b to a patient, wherein the patient is subcutaneously administered a first dose of the antibody on week 0 and a subsequent dose at every 12 weeks thereafter; and wherein the antibody hum13B8-b comprises: (i) a light chain polypeptide comprising the amino acid sequence of SEQ ID NO: 1; and (ii) a heavy chain polypeptide comprising the amino acid sequence of SEQ ID NO: 2; and wherein at least a 75% improvement from baseline value in Psoriasis Area and Severity Index at week 52 indicates the efficacy of the antibody. In some embodiments, at least 90% improvement from baseline value in Psoriasis Area and Severity Index at week 52 indicates the efficacy of the antibody. In some embodiments, a 100% improvement from baseline value in Psoriasis Area and Severity Index at week 52 indicates the efficacy of the antibody.

In some embodiments provided herein is a method of determining the efficacy of an anti-IL-23p19 antibody for the treatment of psoriatic arthritis comprising administering an anti-IL-23p19 antibody hum13B8-b to a patient, wherein the patient is subcutaneously administered a first dose of the antibody on week 0 and a subsequent dose at every 12 weeks thereafter; and wherein the antibody hum13B8-b comprises: (i) a light chain polypeptide comprising the amino acid sequence of SEQ ID NO: 1; and (ii) a heavy chain polypeptide comprising the amino acid sequence of SEQ ID NO: 2; and wherein a reduced DAS28-CRP score from baseline value at week 52 indicates the efficacy of the antibody. In some embodiments, the patient may experience a DAS28 score reduced by 1, 2, 3, 4, 5, 6, 7, 8, or 9 units.

In some embodiments provided herein is a method of determining the efficacy of an anti-IL-23p19 antibody for the treatment of psoriatic arthritis comprising administering an anti-IL-23p19 antibody hum13B8-b to a patient, wherein the patient is subcutaneously administered a first dose of the antibody on week 0 and a subsequent dose at every 12 weeks thereafter; and wherein the antibody hum13B8-b comprises: (i) a light chain polypeptide comprising the amino acid sequence of SEQ ID NO: 1; and (ii) a heavy chain polypeptide comprising the amino acid sequence of SEQ ID NO: 2; and wherein a statistically significant improvement in disease activity as determined by the minimal disease activity (MDA) criteria at week 52 indicates the efficacy of the antibody.

### BRIEF DESCRIPTIONS OF THE DRAWINGS

FIG. 1 is a schematic showing the study design. Abbreviations: B/1=Baseline; LTE=long-term extension; mg=milligram; PtGA=Patient Global Assessment; q=every; Tildra=tildrakizumab; Wk=Week.

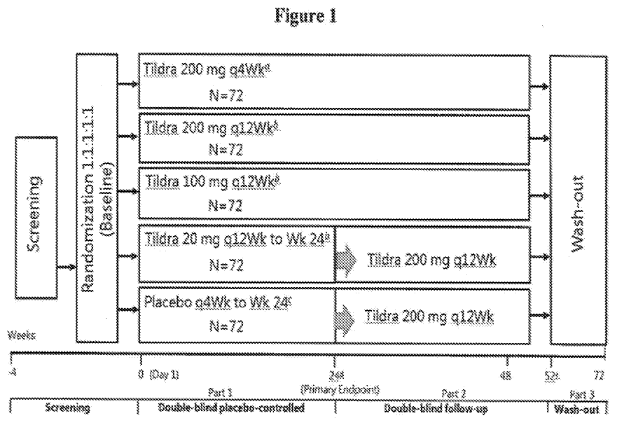

FIG. 2 shows the ACR20/50/70 for patients through week 52 across treatments and time points. Abbreviations: Q4W, every 4 weeks; Q12W, every 12 weeks; TIL, tildrakizumab.

FIG. 3 shows the ACR20/50/70 for patients through week 24 across treatments and time points. Abbreviations: PBO, placebo; Q4W, every 4 weeks; Q12W, every 12 weeks; TIL, tildrakizumab. *P<0.05; †P<0.001; ‡P<0.0001 vs placebo.

FIG. 4 shows minimal disease activity response rates from baseline to week 52 in PsA patients across treatments and time points. Error bars represent the 95% confidence interval. Abbreviations: PsA, psoriatic arthritis; Q4W, every 4 weeks; Q12W, every 12 weeks; TIL, tildrakizumab.

FIG. 5 shows PASI 75/90/100 response rates up to week 52 across treatments and time points. Response rates were calculated in patients with BSA ≥3% at baseline. Black symbols corresponding p-values were analyzed using nonresponse imputation for missing responses. P-values are based on Cochran-Mantel-Haenszel test (with prior anti-TNF use and baseline weight as stratification factors) for nonresponse imputation dataset. *P<0.05; †P<0.001; ‡P<0.0001 versus placebo. Abbreviations: BSA, body surface area; PASI, Psoriasis Area and Severity Index; Q4W, every 4 weeks; Q12W, every 12 weeks; TIL, tildrakizumab.

FIG. 6 shows DAS28-CRP response rates across treatments and time points. Error bars represent 95% confidence interval. Abbreviations: Q4W, every 4 weeks; Q12W, every 12 weeks; TIL, tildrakizumab.

### DETAILED DESCRIPTION

The present disclosure relates to an anti-IL-23p19 antibody hum13B8-b or antigen binding fragment thereof and its use in the treatment of psoriatic arthritis. In some embodiments, provided herein is a method of treating psoriatic arthritis comprising administering an anti-IL-23p19 antibody hum13B8-b to a patient in need thereof, wherein the patient is subcutaneously administered a first dose of the antibody on week 0 and a subsequent dose at every 12 weeks thereafter; and wherein the antibody hum13B8-b comprises: (i) a light chain polypeptide comprising the amino acid sequence of SEQ ID NO: 1; and (ii) a heavy chain polypeptide comprising the amino acid sequence of SEQ ID NO: 2.

As utilized in accordance with the present disclosure, unless otherwise indicated, all technical and scientific terms shall be understood to have the same meaning as commonly understood by one of ordinary skill in the art. Unless otherwise required by context, singular terms shall include pluralities and plural terms shall include the singular.

The term “antibody” as used herein refers to a protein that is capable of recognizing and specifically binding to an antigen. Ordinary or conventional mammalian antibodies comprise a tetramer, which is typically composed of two identical pairs of polypeptide chains, each pair consisting of one “light” chain (typically having a molecular weight of about 25 kDa) and one “heavy” chain (typically having a molecular weight of about 50-70 kDa). The terms “heavy chain” and “light chain,” as used herein, refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. The carboxyl-terminal portion of each chain typically defines a constant domain responsible for effector function. Thus, in a naturally occurring antibody, a full-length heavy chain immunoglobulin polypeptide includes a variable domain (VH) and three constant domains (C_{H1}, C_{H2}, and C_{H3}) and a hinge region between C_{H1} and C_{H2}, wherein the VH domain is at the amino-terminus of the polypeptide and the C_{H3} domain is at the carboxyl-terminus, and a full-length light chain immunoglobulin polypeptide includes a variable domain (V_{L}) and a constant domain (C_{L}), wherein the V_{L} domain is at the amino-terminus of the polypeptide and the C_{L} domain is at the carboxyl-terminus.

Within full-length light and heavy chains, the variable and constant domains typically are joined by a “J” region of about 12 or more amino acids, with the heavy chain also including a “D” region of about 10 more amino acids. The variable regions of each light/heavy chain pair typically form an antigen binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4.

The term “antigen binding fragment” as used herein refers to a portion of an intact antibody and/or refers to the antigenic determining variable domains of an intact antibody. It is known that the antigen binding function of an antibody can be performed by fragments of a full-length antibody. Examples of antibody fragments include, but are not limited to, Fab, Fab′, F(ab′)2, and Fv fragments, linear antibodies, single chain antibodies, diabodies, and multispecific antibodies formed from antibody fragments.

In particular embodiments, the anti-IL-23p19 antibody hum13B8-b is tildrakizumab. The term “tildrakizumab” as used herein refers to a humanized anti-IL-23p19 monoclonal antibody, also known as SCH 900222 or MK-3222. Tildrakizumab is a high-affinity (297 picomolar [pM]) humanized immunoglobulin G1/kappa (IgG1/κ) antibody that specifically binds to the p19 protein of the IL-23 heterodimer but does not bind human IL-12 (IL-12/23p40 and IL12p35 heterodimer) or human IL-12/23p40.

In particular embodiments, tildrakizumab comprises a light chain polypeptide comprising the amino acid sequence of SEQ ID NO: 1 and a heavy chain polypeptide comprising the amino acid sequence of SEQ ID NO: 2, and which is disclosed in U.S. Pat. Nos. 8,404,813 and 8,293,883, the disclosures of each of which are hereby incorporated by reference in their entireties. In other embodiments, tildrakizumab or an antigen-binding fragment thereof comprises a heavy chain variable domain and a light chain variable domain, wherein the heavy chain variable domain comprises CDR1, CDR2, and CDR3 sequences of the amino acid sequences of SEQ ID NOs: 3-5, and wherein the light chain variable domain comprises CDR1, CDR2, and CDR3 sequences of the amino acid sequences of SEQ ID NOs: 6-8.

As used herein, the term “subject” and “patient” are interchangeable. In some embodiments, subjects and/or patients are mammals.

A “disorder” is any condition that would benefit from treatment using the antibodies of the disclosure. “Disorder” and “condition” are used interchangeably herein and include chronic and acute disorders or diseases, including those pathological conditions that predispose a patient to the disorder in question.

The terms “treatment” or “treat” as used herein refer to both therapeutic treatment and prophylactic or preventative measures. Those in need of treatment include patients having psoriatic arthritis as well as those prone to have psoriatic arthritis or those in which psoriatic arthritis is to be prevented.

The terms “administration” or “administering” as used herein refer to providing, contacting, and/or delivering an antibody or fragment thereof by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

In some embodiments, the anti-IL-23p19 antibody hum13B8-b or an antigen-binding fragment thereof is administered every two weeks, every four weeks, every six weeks, every eight weeks, every ten weeks, or every twelve weeks.

As used herein, the term “Week 0” refers to the first day the anti-IL-23p19 antibody hum13B8-b or an antigen-binding fragment thereof is administered.

In some embodiments, the anti-IL-23p19 antibody hum13B8-b or an antigen-binding fragment thereof is administered over a two week treatment period, over a four week treatment period, over a six week treatment period, over an eight week treatment period, over a twelve-week treatment period, over a twenty-four week treatment period, over a thirty-six week treatment period, over a forty-eight week treatment period, over a sixty week treatment period over a seventy-two week treatment period, or over a one year or more treatment period.

The therapy dose of the anti-IL-23p19 antibody hum13B8-b or an antigen-binding fragment thereof will vary depending, in part, upon the size (body weight, body surface, or organ size) and condition (the age and general health) of the patient. In some embodiments, the patient is administered one or more doses of the anti-IL-23p19 antibody hum13B8-b or an antigen-binding fragment thereof wherein the dose is 20 mg, 40 mg, 60 mg, 80 mg, 100 mg, 120 mg, 140 mg, 160 mg, 180 mg, or 200 mg. In some embodiments, the first dose and the subsequent dose of the anti-IL-23p19 antibody hum13B8-b or an antigen-binding fragment thereof are the same. In some embodiments, the first dose and the subsequent dose of the anti-IL-23p19 antibody hum13B8-b or an antigen-binding fragment thereof are different. In some embodiments, the first dose is 100 mg. In some embodiments, the first dose is 200 mg. In some embodiments, the subsequent dose is 100 mg. In some embodiments, the subsequent dose is 200 mg. In some embodiments, the first dose and the subsequent dose are 100 mg. In some embodiments, the first dose and the subsequent dose are 200 mg.

In some embodiments, provided herein is a method of treating psoriatic arthritis comprising administering to a patient in need thereof a therapeutically effective amount of an anti-IL-23p19 antibody hum13B8-b, wherein the treatment results in at least 20%, at least 50%, or at least 70% improvement from baseline value of tender joint count and swollen joint count; and wherein the antibody hum13B8-b comprises: (i) a light chain polypeptide comprising the amino acid sequence of SEQ ID NO: 1; and (ii) a heavy chain polypeptide comprising the amino acid sequence of SEQ ID NO: 2.

For joint counts, five clinical patterns have been described among patients with PsA: distal interphalangeal (DIP), asymmetrical oligoarticular, symmetric polyarticular, spondylitis, and arthritis mutilans. Peripheral joints are assessed for tenderness and swelling. There is no validated measure to assess peripheral joints in PsA; the measure used is the ACR joint count initially developed for the assessment of patients with rheumatoid arthritis (RA). The ACR joint count ranges from 28, 44, 68, and 78 for tenderness, and 28, 44, 66, and 76 for swelling (excluding hips from the assessment of swelling, which cannot be felt at the hip joints). The ACR joint count of 68 tender and 66 swollen joints count includes the majority of joints affected in PsA, and it can be readily performed in a clinic visit. It includes the temporomandibular, sternoclavicular, acromioclavicular, shoulder, elbow, wrist (including the carpometacarpal and intercarpal joints as 1 unit), metacarpophalangeal (MCP), proximal interphalangeal (PIP), DIP, hip, knee, talotibial, midtarsal (including subtalar), metatarsophalangeal, and interphalangeal joints of the toes (proximal and distal joints of each toe is counted as 1 unit).

The American College of Rheumatology 20/50/70 Response Criteria (ACR20/50/70) measures the percentage of subjects with at least a 20%, 50%, or 70% improvement from Baseline in tender joints (68) and swollen joints (66) along with associated percentage improvements in three of five other items: 1) the PGA of disease activity (as measured using a VAS), 2) the PtGA of disease activity (as measured using a VAS), 3) patient pain assessment (as measured using a VAS), 4) patient self-assessed disability (as measured using the HAQ-DI), and 5) acute-phase c-reactive protein (CRP).

C-reactive protein (CRP) or high sensitivity C-reactive protein (hsCRP) is an acute phase reactant, a protein made by the liver and released into the blood within a few hours after tissue injury, the start of an infection, or other cause of inflammation, such as an autoimmune disorder. Markedly increased levels are observed in active Psoriatic Arthritis patients and serves as one of the biomarkers of the Psoriatic Arthritis disease condition.

Physician Global Assessment (PGA) of Disease Activity refers to an assessment wherein a physician evaluates the status of a subject's PsA by means of a visual analog scale (VAS). The subject is assessed according to how the subject's current arthritis is. The VAS is anchored with verbal descriptors of “very good” to “very poor.”

Patient Global Assessment of Disease Activity (PtGA) refers to an assessment wherein a subject assesses their current global status of PsA by means of a VAS (“Considering all the ways your arthritis affects you, on average, how have you been doing today?”), anchored with verbal descriptors of “very well” to “very poorly”.

Patient Pain Assessment refers to an assessment wherein a subject assesses their level of present pain (“How much pain due to your arthritis are you currently experiencing?”) using a VAS. The subject rates their pain at that time on the scale that is anchored with verbal descriptors of “no pain” to “worst possible pain”.

Patient Self-assessed Disability refers to an assessment wherein subjects assess their general disability over the past week using the HAQ-DI questionnaire

In some embodiments, the methods disclosed herein result in at least 20%, at least 50%, or at least 70% improvement from baseline for at least three of the five parameters selected from the group consisting of (i) Physician Global Assessment of disease activity, (ii) Patient Global Assessment of disease activity, (iii) Patient pain assessment, (iv) patient self-assessed disability, and (v) acute-phase CRP.

The Health Assessment Questionnaire-Disability Index (HAQ-DI) is designed to assess patients' usual abilities using their usual equipment. Patients usually find the HAQ-DI self-explanatory, and clarifications are seldom required. There are eight categories assessed by the HAQ-DI: 1) dressing and grooming, 2) arising, 3) eating, 4) walking, 5) hygiene, 6) reach, 7) grip, and 8) common daily activities. For each of these categories, patients report the amount of difficulty they have in performing two or three specific activities. The time frame for the disability questions is the PAST WEEK and each question can be scored as 0 (without any difficulty), 1 (with some difficulty), 2 (with much difficulty) or 3 (unable to do). The use of aids and devices for these activities is also recorded. Use of any device or aid will result in a minimum score of 2 for that category. The score for the disability index is the mean of the eight category scores. If more than two of the categories, or 25%, are missing, the scale is not scored. If fewer than two of the categories are missing, the sum of the categories is divided by the number of answered categories. A higher score indicates greater disability.

The Disease Activity Score 28-item C-Reactive Protein (DAS28-CRP) refers to the measurement of disease activity as assessed across 28 joints including the shoulder, elbow, wrist, MCP (1 through 5), PIP (1 through 5), and knee, with all fourteen joints assessed for each side of the body. It is a composite score derived from examination of the 28 joints for swelling and tenderness, global assessment of pain and overall status using a VAS, and a blood marker of inflammation (hsCRP).

The Leeds dactylometer refers to a validated tool for assessing dactylitis. The dactylometer is used to measure the circumference of the base of the affected digit and is compared to the contralateral digit. The LDI is a measure of this comparison along with a tenderness score (0 =no tenderness, 1 =tender, 2 =tender and wince, and 3 =tender and withdraw) for joints deemed to have dactylitis (where dactylitis is defined as a 10% difference in the ratio of circumference of the affected digit compared to the contralateral digit). The LEI examines tenderness at six sites: two sites (left and right) at each of the lateral epicondyles of the humerus, medial condyles of the femur and the insertion of the Achilles tendon. For each entheseal site, assessment is made of the adjacent joint in terms of tenderness and soft-tissue swelling, with a score of 1 if present. The LEI score range is 0-6.

The Psoriasis Area and Severity Index (PASI) is a measure of the average redness, thickness, and scaliness of the lesions (each graded on a 0-4 scale), weighted by the area of involvement. A 75% reduction in the Psoriasis Area and Severity Index (PASI) score (PASI 75) is a current benchmark of primary endpoints for most clinical trials of psoriasis.

Minimal Disease Activity (MDA) is a measure for disease remission. A patient is classified as having achieved MDA when 5 out of 7 of the following criteria are met: tender joint count ≤1; swollen joint count ≤1; psoriasis activity and severity index ≤1 or body surface area ≤3; patient pain visual analog scale (VAS) score of ≤15; patient global disease activity VAS score of ≤20; Health Assessment Questionnaire (HAQ) score ≤0.5; and tender entheseal points ≤1.

The terms “pharmaceutical composition” or “therapeutic composition” as used herein refer to a compound or composition capable of inducing a desired therapeutic effect when properly administered to a patient. One embodiment of the disclosure provides a pharmaceutical composition comprising a pharmaceutically acceptable carrier and a therapeutically effective amount of at least one antibody of the disclosure.

The terms “pharmaceutically acceptable carrier” or “physiologically acceptable carrier” as used herein refer to one or more formulation materials suitable for accomplishing or enhancing the delivery of one or more antibodies of the disclosure.

Pharmaceutical compositions comprising tildrakizumab, either alone or in combination with prophylactic agents, therapeutic agents, and/or pharmaceutically acceptable carriers are provided. The pharmaceutical compositions comprising tildrakizumab provided herein are for use in, but not limited to, diagnosing, detecting, or monitoring a disorder, in preventing, treating, managing, or ameliorating a disorder or one or more symptoms thereof, and/or in research. The formulation of pharmaceutical compositions, either alone or in combination with prophylactic agents, therapeutic agents, and/or pharmaceutically acceptable carriers, is known to one skilled in the art.

In some embodiments provided herein is a method of determining the efficacy of an anti-IL-23p19 antibody for the treatment of psoriatic arthritis comprising administering an anti-IL-23p19 antibody hum13B8-b to a patient, wherein the patient is subcutaneously administered a first dose of the antibody on week 0 and a subsequent dose at every 12 weeks thereafter; wherein the antibody hum13B8-b comprises: (i) a light chain polypeptide comprising the amino acid sequence of SEQ ID NO: 1; and (ii) a heavy chain polypeptide comprising the amino acid sequence of SEQ ID NO: 2; and wherein an ACR20 response value of at least about 40% at week 24 or week 52 indicates the efficacy of the antibody. In some embodiments, an ACR20 response value of at least about 50% at week 24 or week 52 indicates the efficacy of the antibody. In some embodiments, an ACR20 response value of at least about 60% at week 24 or week 52 indicates the efficacy of the antibody.

In some embodiments provided herein is a method of determining the efficacy of an anti-IL-23p19 antibody for the treatment of psoriatic arthritis comprising administering an anti-IL-23p19 antibody hum13B8-b to a patient, wherein the patient is subcutaneously administered a first dose of the antibody on week 0 and a subsequent dose at every 12 weeks thereafter; and wherein the antibody hum13B8-b comprises: (i) a light chain polypeptide comprising the amino acid sequence of SEQ ID NO: 1; and (ii) a heavy chain polypeptide comprising the amino acid sequence of SEQ ID NO: 2; and wherein an ACR50 response value of at least about 20% at week 24 or week 52 indicates the efficacy of the antibody. In some embodiments, an ACR50 response value of at least about 25% at week 24 or week 52 indicates the efficacy of the antibody. In some embodiments, an ACR50 response value of at least about 30% at week 24 or week 52 indicates the efficacy of the antibody.

In some embodiments provided herein is a method of determining the efficacy of an anti-IL-23p19 antibody for the treatment of psoriatic arthritis comprising administering an anti-IL-23p19 antibody hum13B8-b to a patient, wherein the patient is subcutaneously administered a first dose of the antibody on week 0 and a subsequent dose at every 12 weeks thereafter; and wherein the antibody hum 13B8-b comprises: (i) a light chain polypeptide comprising the amino acid sequence of SEQ ID NO: 1; and (ii) a heavy chain polypeptide comprising the amino acid sequence of SEQ ID NO: 2; and wherein an ACR70 response value of at least about 10% at week 24 or week 52 indicates the efficacy of the antibody. In some embodiments, an ACR70 response value of at least about 12% at week 24 indicates the efficacy of the antibody. In some embodiments, an ACR70 response value of at least about 15% at week 24 or week 52 indicates the efficacy of the antibody.

In some embodiments provided herein is a method of determining the efficacy of an anti-IL-23p19 antibody for the treatment of psoriatic arthritis comprising administering an anti-IL-23p19 antibody hum13B8-b to a patient, wherein the patient is subcutaneously administered a first dose of the antibody on week 0 and a subsequent dose at every 12 weeks thereafter; and wherein the antibody hum13B8-b comprises: (i) a light chain polypeptide comprising the amino acid sequence of SEQ ID NO: 1; and (ii) a heavy chain polypeptide comprising the amino acid sequence of SEQ ID NO: 2; and wherein at least a 75% improvement from baseline value in Psoriasis Area and Severity Index at week 52 indicates the efficacy of the antibody. In some embodiments, at least 90% improvement from baseline value in Psoriasis Area and Severity Index at week 52 indicates the efficacy of the antibody. In some embodiments, a 100% improvement from baseline value in Psoriasis Area and Severity Index at week 52 indicates the efficacy of the antibody.

In some embodiments provided herein is a method of determining the efficacy of an anti-IL-23p19 antibody for the treatment of psoriatic arthritis comprising administering an anti-IL-23p19 antibody hum13B8-b to a patient, wherein the patient is subcutaneously administered a first dose of the antibody on week 0 and a subsequent dose at every 12 weeks thereafter; and wherein the antibody hum13B8-b comprises: (i) a light chain polypeptide comprising the amino acid sequence of SEQ ID NO: 1; and (ii) a heavy chain polypeptide comprising the amino acid sequence of SEQ ID NO: 2; and wherein a reduced DAS28-CRP score from baseline value at week 52 indicates the efficacy of the antibody. In some embodiments, the patient may experience a DAS28 score reduced by 1, 2, 3, 4, 5, 6, 7, 8, or 9 units.

In some embodiments provided herein is a method of determining the efficacy of an anti-IL-23p19 antibody for the treatment of psoriatic arthritis comprising administering an anti-IL-23p19 antibody hum13B8-b to a patient, wherein the patient is subcutaneously administered a first dose of the antibody on week 0 and a subsequent dose at every 12 weeks thereafter; and wherein the antibody hum 13B8-b comprises: (i) a light chain polypeptide comprising the amino acid sequence of SEQ ID NO: 1; and (ii) a heavy chain polypeptide comprising the amino acid sequence of SEQ ID NO: 2; and wherein a statistically significant improvement in disease activity as determined by the minimal disease activity (MDA) criteria at week 52 indicates the efficacy of the antibody.

### EXAMPLES

The Examples that follow are illustrative of specific embodiments of the disclosure, and various uses thereof They are set forth for explanatory purposes only and should not be construed as limiting the scope of the disclosure in any way.

### Example 1: Administration of Tildrakizumab in Subjects with Active Psoriatic Arthritis

### 1. Design of the Study

A randomized, double-blind, placebo-controlled, multiple-dose, Phase 2b study was conducted to evaluate the efficacy of four dose groups of tildrakizumab administered by subcutaenous injection in subjects with active PsA (NCT02980692). Subjects with active PsA were randomized 1:1:1:1:1 to receive 200 milligram (mg) tildrakizumab administered by subcutaneous (SC) injection every (q) 4 weeks up until Week 52, 200 mg tildrakizumab administered SC q12 weeks up until Week 52, 100 mg tildrakizumab administered SC q12 weeks up until Week 52, 20 mg tildrakizumab administered SC at Weeks 0 and 12, then tildrakizumab 200 mg at Weeks 24 and q12 weeks thereafter up until Week 52, or placebo administered SC at Weeks 0, 4, 8, 12, 16, 20, and 24, and then tildrakizumab 200 mg q12 weeks thereafter up until Week 52. All subjects received injections q4 weeks; subjects randomized to the 12-weekly active treatment groups received placebo injections at Weeks 4, 8, 16, 20, 28, 32, 40, and 44.

The study consisted of a Screening Period (Days -28 to 0); Part 1, a double-blind, placebo-controlled period (Day 1 to Week 24); Part 2, a double-blind follow-up period (Week 25 to 52); and Part 3, a 20-week wash-out period (Week 53 to 72). During the wash-out period, subjects no longer received tildrakizumab. Subjects who showed clinical response to treatment (defined as >20% improvement from Baseline in both the swollen and tender joint counts and >20% improvement from Baseline in the Patient Global Assessment [PtGA] of disease activity) at Week 24 entered Part 2 of the study. Subjects receiving tildrakizumab (100 mg q12 weeks or 200 mg [q4 and q12 weeks] dose groups) during Part 1 who failed to show clinical response to treatment at Week 24 were discontinued from the study drug. Subjects receiving placebo or 20 mg tildrakizumab during Part 1 who failed to show clinical response to treatment at Week 24 entered Part 2 and received 200 mg tildrakizumab q12 weeks until Week 52. Subjects in Part 2 who were not deriving sufficient clinical benefit at any time after Week 24, were discontinued from study drug.

The primary endpoint was measured by the proportion of subjects achieving 20% improvement from Baseline in American College of Rheumatology response criteria (ACR20) response rate at Week 24 and 52. Secondary efficacy endpoints included ACR50, ACR70 response rates, and the components of ACR response; proportion of subjects who require adjustment of background therapy; proportion of subjects who achieved a DAS28-CRP <3.2; proportion of subjects who achieved minimal disease activity (MDA) criteria; Leeds Dactylitis Index (LDI) and Leeds Enthesitis Index (LEI) change from Baseline; HAQ-DI change from Baseline, and 75%/90%/100% improvement in the Psoriasis Area and Severity Index (PASI). The PK and immunogenicity of tildrakizumab and Treatment-emergent adverse events (TEAEs) were also be evaluated.

### 2. Selection of Study Population

The subject population included subjects ≥18 years of age, with a diagnosis of PsA (by the Classification of Psoriatic Arthritis [CASPAR] criteria) with symptoms present for at least 6 months, ≥3 tender and ≥3 swollen joints at Screening and Baseline.

Subjects were excluded from participation in the study if aspartate aminotransferase (AST) or alanine aminotransferase (ALT) ≥2 times upper limit of normal (ULN), creatinine ≥1.5 times the ULN, serum direct bilirubin ≥1.5 mg/dL, white blood cell (WBC) count ≥3.0×103/μL, or positive test result for rheumatoid factor.

Table 1 provides a summary of the demographic characteristics by treatment for the selected subjects. Table 2 provides a summary of the baseline disease characteristics for the selected subjects. N=number of subjects in the treatment group analysis set and n=number of subjects in the specified category with non-missing values. Baseline is defined as the last available value before the first dose of study drug.

TABLE 1
Summary of Demographic Characteristics by Treatment Full Analysis Set

| | 200 mg | | 200 mg | | | | 20 mg −> | | Placebo −> | | | |
|---|---|---|---|---|---|---|---|---|---|---|---|---|
| | Tildrakizumab | | Tildrakizumab | | 100 mg | | 200 mg | | 200 mg | | | |
| Charac- | q4wk | | q12wk | | Tildrakizumab | | Tildrakizumab | | Tildrakizumab | | Total | |
| teristic | (N = 78) | | (N = 79) | | (N = 77) | | (N = 78) | | (N = 79) | | (N = 391) | |
| Age (years) | | | | | | | | | | | | |
| N | 78 | | 79 | | 77 | | 78 | | 79 | | 391 | |
| Mean (SD) | 50.1 | (13.28) | 49.3 | (11.24) | 49.2 | (11.85) | 47.2 | (13.35) | 48.1 | (13.30) | 48.8 | (12.61) |
| Median | 50.0 | | 49.0 | | 50.0 | | 47.5 | | 47.0 | | 48.0 | |
| Min, Max | 20, 90 | | 26, 71 | | 18, 77 | | 18, 71 | | 18, 75 | | 18, 90 | |
| Sex n (%) | | | | | | | | | | | | |
| Male | 32 | (41.0) | 42 | (53.2) | 30 | (39.0) | 37 | (47.4) | 35 | (44.3) | 176 | (45.0) |
| Female | 46 | (59.0) | 37 | (46.8) | 47 | (61.0) | 41 | (52.6) | 44 | (55.7) | 215 | (55.0) |
| Race n (%) | | | | | | | | | | | | |
| White | 76 | (97.4) | 78 | (98.7) | 75 | (97.4) | 75 | (96.2) | 74 | (93.7) | 378 | (96.7) |
| Black or | 0 | | 0 | | 1 | (1.3) | 1 | (1.3) | 3 | (3.8) | 5 | (1.3) |
| African | | | | | | | | | | | | |
| American | | | | | | | | | | | | |
| Asian | 0 | | 0 | | 0 | | 0 | | 0 | | 0 | |
| Other | 2 | (2.6) | 1 | (1.3) | 1 | (1.3) | 2 | (2.6) | 2 | (2.5) | 8 | (2.0) |
| Ethnicity n (%) | | | | | | | | | | | | |
| Hispanic/ | 12 | (15.4) | 16 | (20.3) | 11 | (14.3) | 9 | (11.5) | 11 | (13.9) | 59 | (15.1) |
| Latino | | | | | | | | | | | | |
| Not Hispanic/ | 66 | (84.6) | 63 | (79.7) | 66 | (85.7) | 69 | (88.5) | 68 | (86.1) | 332 | (84.9) |
| Latino | | | | | | | | | | | | |
| Height (cm) | | | | | | | | | | | | |
| N | 78 | | 79 | | 77 | | 78 | | 79 | | 391 | |
| Mean (SD) | 167.95 | (8.944) | 169.93 | (9.930) | 168.54 | (8.921) | 169.99 | (10.064) | 169.86 | (9.896) | 169.26 | (9.556) |
| Median | 168.00 | | 170.00 | | 168.00 | | 168.46 | | 170.00 | | 168.00 | |
| Min, Max | 150.0, 189.0 | | 140.0, 190.0 | | 150.5, 191.8 | | 151.3, 195.0 | | 147.2, 189.0 | | 140.0, 195.0 | |
| Weight (kg) | | | | | | | | | | | | |
| N | 78 | | 79 | | 77 | | 78 | | 79 | | 391 | |
| Mean (SD) | 85.05 | (19.690) | 87.09 | (19.513) | 83.59 | (18.904) | 85.13 | (18.109) | 85.31 | (20.213) | 85.24 | (19.236) |
| Median | 85.70 | | 85.00 | | 84.30 | | 84.25 | | 83.00 | | 85.00 | |
| Min, Max | 44.5, 135.0 | | 48.7, 157.8 | | 52.0, 147.0 | | 50.4, 142.5 | | 50.0, 140.3 | | 44.5, 157.8 | |
| BMI (kg/m**2) | | | | | | | | | | | | |
| N | 78 | | 79 | | 77 | | 78 | | 79 | | 391 | |
| Mean (SD) | 30.11 | (6.523) | 30.19 | (6.462) | 29.48 | (6.846) | 29.37 | (5.192) | 29.46 | (5.958) | 29.72 | (6.199) |

| | | | | | | |
|---|---|---|---|---|---|---|
| Median | 30.01 | 29.31 | 27.84 | 28.89 | 27.79 | 28.91 |
| Min, Max | 17.8, 49.5 | 18.6, 51.4 | 20.6, 50.8 | 18.1, 43.6 | 19.3, 46.3 | 17.8, 51.4 |

Abbreviations: q = every; wk = week; BMI = body mass index; SD = Standard Deviation; Min = Minimum; Max = Maximum.

TABLE 2
Baseline Disease Characteristics Full Analysis Set

| | 200 mg | 200 mg | | 20 mg −> | Placebo −> | |
|---|---|---|---|---|---|---|
| | Tildrakizumab | Tildrakizumab | 100 mg | 200 mg | 200 mg | |
| | q4wk | q12wk | Tildrakizumab | Tildrakizumab | Tildrakizumab | Total |
| Characteristics | (N = 78) | (N = 79) | (N = 77) | (N = 78) | (N = 79) | (N = 391) |
| Baseline Rheumatoid Factor | | | | | | |
| (IU/mL) | | | | | | |
| N | 77 | 79 | 77 | 75 | 79 | 387 |
| Mean | 20.00 | 20.00 | 21.40 | 20.60 | 24.53 | 21.32 |
| SD | 0.000 | 0.000 | 9.242 | 5.173 | 23.333 | 11.614 |
| Median | 20.00 | 20.00 | 20.00 | 20.00 | 20.00 | 20.00 |
| Min, Max | 20.0, 20.0 | 20.0, 20.0 | 20.0, 95.2 | 20.0, 64.8 | 20.0, 168.8 | 20.0, 168.8 |
| Missing | 1 | 0 | 0 | 3 | 0 | 4 |
| Baseline BSA Affected (%) | | | | | | |
| N | 78 | 79 | 77 | 78 | 79 | 391 |
| Mean | 11.9 | 9.0 | 12.8 | 10.4 | 8.2 | 10.4 |
| SD | 16.02 | 12.38 | 16.01 | 14.11 | 12.18 | 14.26 |
| Median | 5.0 | 4.0 | 8.0 | 4.0 | 3.0 | 5.0 |
| Min, Max | 0, 85 | 0, 56 | 0, 90 | 0, 70 | 0, 80 | 0, 90 |
| Baseline BSA | | | | | | |
| Affected >=3% n(%) | | | | | | |
| Yes | 53 (67.9) | 44 (55.7) | 54 (70.1) | 41 (52.6) | 42 (53.2) | 234 (59.8) |
| No | 25 (32.1) | 35 (44.3) | 23 (29.9) | 37 (47.4) | 37 (46.8) | 157 (40.2) |
| Baseline Tendor Joint Counts | | | | | | |
| N | 78 | 79 | 77 | 78 | 79 | 391 |
| Mean | 16.6 | 19.5 | 21.3 | 19.0 | 19.7 | 19.2 |
| SD | 11.93 | 13.90 | 14.80 | 12.95 | 14.66 | 13.70 |
| Median | 13.5 | 15.0 | 19.0 | 14.0 | 15.0 | 15.0 |
| Min, Max | 3, 64 | 4, 63 | 3, 59 | 4, 54 | 3, 64 | 3, 64 |
| Baseline Swollen Joint Counts | | | | | | |
| N | 78 | 79 | 77 | 78 | 79 | 391 |
| Mean | 10.4 | 10.0 | 11.0 | 9.4 | 11.8 | 10.5 |
| SD | 7.43 | 7.95 | 8.21 | 6.41 | 9.75 | 8.03 |
| Median | 8.0 | 7.0 | 8.0 | 8.0 | 8.0 | 8.0 |
| Min, Max | 3, 35 | 3, 45 | 0, 38 | 3, 38 | 3, 42 | 0, 45 |
| Baseline PGA of Disease | | | | | | |
| Activity Score | | | | | | |
| N | 78 | 79 | 77 | 78 | 79 | 391 |
| Mean | 54.0 | 55.4 | 57.3 | 59.4 | 59.5 | 57.1 |
| SD | 16.12 | 16.21 | 17.31 | 14.44 | 15.59 | 16.02 |
| Median | 55.5 | 56.0 | 59.0 | 60.5 | 58.0 | 57.0 |
| Min, Max | 9, 88 | 20, 83 | 3, 95 | 25, 94 | 19, 93 | 3, 95 |
| Baseline PtGA of Disease | | | | | | |
| Activity Score | | | | | | |
| N | 78 | 79 | 77 | 78 | 79 | 391 |
| Mean | 57.8 | 61.1 | 60.3 | 61.9 | 65.2 | 61.3 |
| SD | 18.31 | 20.74 | 20.24 | 17.36 | 18.12 | 19.05 |
| Median | 57.5 | 66.0 | 65.0 | 62.0 | 66.0 | 62.0 |
| Min, Max | 13, 90 | 5, 94 | 16, 100 | 21, 100 | 21, 100 | 5, 100 |
| Baseline Patient's Pain | | | | | | |
| Assessment Score | | | | | | |
| N | 78 | 79 | 77 | 78 | 79 | 391 |
| Mean | 55.4 | 59.6 | 59.2 | 60.9 | 64.2 | 59.9 |
| SD | 19.09 | 23.54 | 22.08 | 19.70 | 20.36 | 21.10 |
| Median | 57.0 | 65.0 | 60.0 | 63.0 | 68.0 | 62.0 |
| Min, Max | 9, 97 | 4, 98 | 5, 100 | 16, 100 | 13, 100 | 4, 100 |
| Baseline HAQ-DI | | | | | | |
| N | 78 | 79 | 77 | 78 | 79 | 391 |
| Mean | 1.0481 | 1.0111 | 1.0471 | 1.0545 | 1.1614 | 1.0646 |
| SD | 0.61806 | 0.64307 | 0.71031 | 0.60255 | 0.59584 | 0.63384 |
| Median | 1.1250 | 1.0000 | 1.1250 | 1.0000 | 1.2500 | 1.1250 |
| Min, Max | 0.000, 2.875 | 0.000, 2.500 | 0.000, 2.750 | 0.000, 2.375 | 0.000, 2.500 | 0.000, 2.875 |
| Baseline hsCRP (mg/L) | | | | | | |
| N | 78 | 79 | 77 | 78 | 79 | 391 |
| Mean | 7.827 | 10.483 | 10.587 | 10.733 | 13.002 | 10.533 |
| SD | 18.6437 | 14.4210 | 20.0102 | 13.9524 | 20.8016 | 17.7744 |
| Median | 3.310 | 3.720 | 4.880 | 5.145 | 5.720 | 4.390 |
| Min, Max | 0.30, 156.50 | 0.27, 85.04 | 0.10, 155.87 | 0.22, 67.43 | 0.21, 123.95 | 0.10, 156.50 |

Abbreviations: BSA = Body Surface Area; hsCRP = C-reactive protein; HAQ-DI = Health Assessment Questionnaire Disability Index; PGA = Physician Global Assessment; PtGA = Patient Global Assessment; q = every; wk = week; SD = Standard Deviation; Min = Minimum; Max = Maximum.

### 3. Statistical Analysis

The primary efficacy analysis population was the Full Analysis Set (FAS) defined as all randomized subjects who have received at least 1 dose of Investigational Medicinal Product (IMP). The primary analysis was based on the Cochran-Mantel-Haenszel test, incorporating prior anti-TNF use and Baseline weight as stratification factors, to compare response rates for the primary endpoint (ACR20 at Week 24 and Week 52) between placebo and each of the respective active dose arms. In addition, response rate difference between placebo and each of the respective active dose arms and the corresponding confidence interval (CI) was estimated. Early withdrawals and any other subjects with incomplete data at Week 24 were classified as non-responders for the primary endpoint (ACR20). Subjects who failed to show minimal response to treatment (defined as <10% improvement from Baseline in either swollen or tender joint counts) at Week 16 may have had their background medications adjusted according to the maximum permitted daily dose and continue in the study. Any subjects requiring these adjustments were counted as non-responders for the primary analysis.

Analyses of the primary endpoint will be based on the FAS. A sensitivity analysis was performed based on PPAS.

### 4. Results up to Week 24

The summary of the subject status at Week 24 is presented in Table 3. The safety analysis set consisted of all randomized subjects who received at least 1 dose of IMP. The full analysis set consisted of all randomized subjects who received at least 1 dose of investigational medicinal product (IMP). The per-protocol analysis set consisted of all subjects in the full analysis set without any major protocol deviations that could have influenced the validity of the data for the primary efficacy variables. Percentages were based on the number of subjects randomized except for Completers and Discontinuation where percentages are based on the number of subjects in the safety analysis set. Part 1 is a double-blind placebo-controlled period from baseline to Week 24, and Part 2 is a double-blind follow-up period from Week 25 to Week 52.

TABLE 3
Summary of Subject Status at Week 24

| | 200 mg | | 200 mg | | | | 20 mg −> | | Placebo -> | | | |
|---|---|---|---|---|---|---|---|---|---|---|---|---|
| | Tildrakizumab | | Tildrakizumab | | 100 mg | | 200 mg | | 200 mg | | | |
| | q4wk | | q12wk | | Tildrakizumab | | Tildrakizumab | | Tildrakizumab | | Total | |
| Population Subset | n(%) | | n(%) | | n(%) | | n(%) | | n(%) | | n(%) | |
| Number of Subject Screened | | | | | | | | | | | 500 | |
| Number of Screen Failure | | | | | | | | | | | 109 | |
| Number of Subject Randomized | 78 | | 79 | | 77 | | 78 | | 79 | | 391 | |
| Did not take study drug | 0 | | 0 | | 0 | | 0 | | 0 | | 0 | |
| Safety Analysis Set (a) | 78 | (100.0) | 79 | (100.0) | 77 | (100.0) | 78 | (100.0) | 79 | (100.0) | 391 | (100.0) |
| Full Analysis Set (b) | 78 | (100.0) | 79 | (100.0) | 77 | (100.0) | 78 | (100.0) | 79 | (100.0) | 391 | (100.0) |
| Per Protocol Analysis Set (c) | 78 | (100.0) | 79 | (100.0) | 77 | (100.0) | 78 | (100.0) | 79 | (100.0) | 391 | (100.0) |
| Completers for Treatment | 5 | (6.4) | 2 | (2.5) | 5 | (6.5) | 5 | (6.4) | 9 | (11.4) | 26 | (6.6) |
| Discontinued from Treatment | 16 | (20.5) | 17 | (21.5) | 21 | (27.3) | 7 | (9.0) | 7 | (8.9) | 68 | (17.4) |
| Completed Part 1 Treatment | 61 | (78.2) | 64 | (81.0) | 60 | (77.9) | 71 | (91.0) | 74 | (93.7) | 330 | (84.4) |
| Discontinued Treatment in Part 1 | 16 | (20.5) | 15 | (19.0) | 17 | (22.1) | 7 | (9.0) | 5 | (6.3) | 60 | (15.3) |
| Completed Part 2 Treatment | 5 | (6.4) | 2 | (2.5) | 5 | (6.5) | 5 | (6.4) | 9 | (11.4) | 26 | (6.6) |
| Discontinued Treatment in Part 2 | 0 | | 2 | (2.5) | 4 | (5.2) | 0 | | 2 | (2.5) | 8 | (2.0) |
| Completers for Study | 8 | (10.3) | 6 | (7.6) | 9 | (11.7) | 6 | (7.7) | 8 | (10.1) | 37 | (9.5) |
| Discontinued from Study | 7 | (9.0) | 5 | (6.3) | 11 | (14.3) | 4 | (5.1) | 6 | (7.6) | 33 | (8.4) |

Abbreviations: q = every; wk = week; IMP = Investigational Medicinal Product.

The Cochran-Mantel Haenszel (CMH) analysis of ACR20 response rates up to Week 24 is illustrated in Table 4. ACR20 was calculated as a >=20% improvement from baseline in tender and swollen joint counts and >=20% improvement from baseline in three of the five remaining ACR-core set measures: patient and physician global assessments, pain, disability, and an acute-phase CRP. Subjects receiving tildrakizumab (100 mg q12 weeks or 200 mg [q4 and q12 weeks] dose groups) during Part 1 who failed to show clinical response to treatment at Week 24 were discontinued study drug and entered the washout period per protocol. Week 24 assessments for subjects who discontinue study drug were recorded at Week 52/EOT. Two-sided 95% CI and p-value were based on the CMH test with prior anti-TNF use and Baseline weight as stratification factors. If Mantel-Fleiss criterion was less than 5, pairwise comparisons were based on Fisher's exact tests after collapsing across levels of the stratification factors. This is noted with a “*” for p-value. Baseline is defined as the last available value before the first dose of study drug.

TABLE 4
CMH Analysis of ACR20 Response Rates up to Week 24 (Missing
Response = Non-response) -Primary Analysis Full Analysis Set

| | | | | | | Comparison to Placebo | | | | |
|---|---|---|---|---|---|---|---|---|---|---|
| | | | | | | | | 95% Confidence | | |
| | | | | | | | SE of | Interval | | |
| | | | | Response | SE | Difference | Difference | Lower | Upper | |
| Visit | Treatment Group | N | n | Rate(%) | (%) | (%) | (%) | (%) | (%) | p-value |
| Week 1 | 200 mg tildrakizumab | 78 | 8 | 10.26 | 3.44 | | | | | |
| | q4wk | | | | | | | | | |
| | 200 mg tildrakizumab | 79 | 9 | 11.39 | 3.57 | | | | | |
| | q12wk | | | | | | | | | |
| | 100 mg tildrakizumab | 77 | 5 | 6.49 | 2.81 | | | | | |
| | 20 mg tildrakizumab | 78 | 5 | 6.41 | 2.77 | | | | | |
| | Placebo | 79 | 4 | 5.06 | 2.47 | | | | | |
| | 200 mg tildrakizumab | | | | | 5.18 | 4.20 | −3.05 | 13.41 | 0.2235 |
| | q4wk vs Placebo | | | | | | | | | |
| | 200 mg tildrakizumab | | | | | 6.34 | 4.35 | −2.18 | 14.86 | 0.1504 |
| | q12wk vs Placebo | | | | | | | | | |
| | 100 mg tildrakizumab | | | | | 1.43 | 3.74 | −5.90 | 8.76 | 0.7441* |
| | vs Placebo | | | | | | | | | |
| | 20 mg tildrakizumab | | | | | 1.35 | 3.71 | −5.93 | 8.62 | 0.7458* |
| | vs Placebo | | | | | | | | | |
| Week 4 | 200 mg tildrakizumab | 78 | 17 | 21.79 | 4.67 | | | | | |
| | q4wk | | | | | | | | | |
| | 200 mg tildrakizumab | 79 | 16 | 20.25 | 4.52 | | | | | |
| | q12wk | | | | | | | | | |
| | 100 mg tildrakizumab | 77 | 23 | 29.87 | 5.22 | | | | | |
| | 20 mg tildrakizumab | 78 | 14 | 17.95 | 4.35 | | | | | |
| | Placebo | 79 | 15 | 18.99 | 4.41 | | | | | |
| | 200 mg tildrakizumab | | | | | 2.85 | 6.31 | −9.53 | 15.22 | 0.6553 |
| | q4wk vs Placebo | | | | | | | | | |
| | 200 mg tildrakizumab | | | | | 1.31 | 6.38 | −11.19 | 13.81 | 0.8378 |
| | q12wk vs Placebo | | | | | | | | | |
| | 100 mg tildrakizumab | | | | | 11.04 | 6.90 | −2.49 | 24.57 | 0.1125 |
| | vs Placebo | | | | | | | | | |
| | 20 mg tildrakizumab | | | | | −0.96 | 6.27 | −13.25 | 11.32 | 0.8777 |
| | vs Placebo | | | | | | | | | |
| Week 8 | 200 mg tildrakizumab | 78 | 35 | 44.87 | 5.63 | | | | | |
| | q4wk | | | | | | | | | |
| | 200 mg tildrakizumab | 79 | 30 | 37.97 | 5.46 | | | | | |
| | q12wk | | | | | | | | | |
| | 100 mg tildrakizumab | 77 | 37 | 48.05 | 5.69 | | | | | |
| | 20 mg tildrakizumab | 78 | 29 | 37.18 | 5.47 | | | | | |
| | Placebo | 79 | 22 | 27.85 | 5.04 | | | | | |
| | 200 mg tildrakizumab | | | | | 16.89 | 7.49 | 2.22 | 31.56 | 0.0261 |
| | q4wk vs Placebo | | | | | | | | | |
| | 200 mg tildrakizumab | | | | | 10.09 | 7.48 | −4.57 | 24.75 | 0.1812 |
| | q12wk vs Placebo | | | | | | | | | |
| | 100 mg tildrakizumab | | | | | 20.49 | 7.53 | 5.74 | 35.24 | 0.0086 |
| | vs Placebo | | | | | | | | | |
| | 20 mg tildrakizumab | | | | | 9.46 | 7.47 | −5.18 | 24.09 | 0.2091 |
| | vs Placebo | | | | | | | | | |
| Week 12 | 200 mg tildrakizumab | 78 | 41 | 52.56 | 5.65 | | | | | |
| | q4wk | | | | | | | | | |
| | 200 mg tildrakizumab | 79 | 39 | 49.37 | 5.62 | | | | | |
| | q12wk | | | | | | | | | |
| | 100 mg tildrakizumab | 77 | 36 | 46.75 | 5.69 | | | | | |
| | 20 mg tildrakizumab | 78 | 32 | 41.03 | 5.57 | | | | | |
| | Placebo | 79 | 26 | 32.91 | 5.29 | | | | | |
| | 200 mg tildrakizumab | | | | | 19.37 | 7.68 | 4.31 | 34.43 | 0.0144 |
| | q4wk vs Placebo | | | | | | | | | |
| | 200 mg tildrakizumab | | | | | 16.25 | 7.73 | 1.11 | 31.39 | 0.0395 |
| | q12wk vs Placebo | | | | | | | | | |
| | 100 mg tildrakizumab | | | | | 13.90 | 7.76 | −1.30 | 29.11 | 0.0759 |
| | vs Placebo | | | | | | | | | |
| | 20 mg tildrakizumab | | | | | 8.03 | 7.69 | −7.05 | 23.11 | 0.3019 |
| | vs Placebo | | | | | | | | | |
| Week 16 | 200 mg tildrakizumab | 78 | 54 | 69.23 | 5.23 | | | | | |
| | q4wk | | | | | | | | | |
| | 200 mg tildrakizumab | 79 | 45 | 56.96 | 5.57 | | | | | |
| | q12wk | | | | | | | | | |
| | 100 mg tildrakizumab | 77 | 42 | 54.55 | 5.67 | | | | | |
| | 20 mg tildrakizumab | 78 | 36 | 46.15 | 5.64 | | | | | |
| | Placebo | 79 | 27 | 34.18 | 5.34 | | | | | |
| | 200 mg tildrakizumab | | | | | 34.51 | 7.39 | 20.02 | 48.99 | <.0001 |
| | q4wk vs Placebo | | | | | | | | | |
| | 200 mg tildrakizumab | | | | | 22.34 | 7.65 | 7.34 | 37.34 | 0.0047 |
| | q12wk vs Placebo | | | | | | | | | |
| | 100 mg tildrakizumab | | | | | 20.34 | 7.79 | 5.07 | 35.61 | 0.0114 |
| | vs Placebo | | | | | | | | | |
| | 20 mg tildrakizumab | | | | | 11.81 | 7.76 | −3.40 | 27.03 | 0.1349 |
| | vs Placebo | | | | | | | | | |
| Week 20 | 200 mg tildrakizumab | 78 | 57 | 73.08 | 5.02 | | | | | |
| | q4wk | | | | | | | | | |
| | 200 mg tildrakizumab | 79 | 54 | 68.35 | 5.23 | | | | | |
| | q12wk | | | | | | | | | |
| | 100 mg tildrakizumab | 77 | 46 | 59.74 | 5.59 | | | | | |
| | 20 mg tildrakizumab | 78 | 42 | 53.85 | 5.64 | | | | | |
| | Placebo | 79 | 28 | 35.44 | 5.38 | | | | | |
| | 200 mg tildrakizumab | | | | | 37.19 | 7.29 | 22.90 | 51.48 | <.0001 |
| | q4wk vs Placebo | | | | | | | | | |
| | 200 mg tildrakizumab | | | | | 32.71 | 7.49 | 18.03 | 47.39 | <.0001 |
| | q12wk vs Placebo | | | | | | | | | |
| | 100 mg tildrakizumab | | | | | 24.05 | 7.69 | 8.99 | 39.11 | 0.0026 |
| | vs Placebo | | | | | | | | | |
| | 20 mg tildrakizumab | | | | | 18.09 | 7.75 | 2.89 | 33.29 | 0.0228 |
| | vs Placebo | | | | | | | | | |
| Week 24 | 200 mg tildrakizumab | 78 | 62 | 79.49 | 4.57 | | | | | |
| | q4wk | | | | | | | | | |
| | 200 mg tildrakizumab | 79 | 61 | 77.22 | 4.72 | | | | | |
| | q12wk | | | | | | | | | |
| | 100 mg tildrakizumab | 77 | 55 | 71.43 | 5.15 | | | | | |
| | 20 mg tildrakizumab | 78 | 57 | 73.08 | 5.02 | | | | | |
| | Placebo | 79 | 40 | 50.63 | 5.62 | | | | | |
| | 200 mg tildrakizumab | | | | | 28.42 | 7.16 | 14.39 | 42.45 | 0.0002 |
| | q4wk vs Placebo | | | | | | | | | |
| | 200 mg tildrakizumab | | | | | 26.22 | 7.33 | 11.85 | 40.58 | 0.0006 |
| | q12wk vs Placebo | | | | | | | | | |
| | 100 mg tildrakizumab | | | | | 20.54 | 7.54 | 5.75 | 35.32 | 0.0085 |
| | vs Placebo | | | | | | | | | |
| | 20 mg tildrakizumab | | | | | 22.21 | 7.53 | 7.44 | 36.98 | 0.0043 |
| | vs Placebo | | | | | | | | | |

Abbreviations: q = every; wk = week; ACR = American College of Rheumatology; CMH = Cochran-Mantel-Haenszel; CRP = C-reactive protein; SE = Standard Error.
N = number of subjects in full analysis set.
n = number of responders.

Table 5 illustrates CMH analysis of the ACR 50 response rates up to Week 24. ACR50 is calculated as a >=50% improvement from baseline in tender and swollen joint counts and >=50% improvement from baseline in three of the five remaining ACR-core set measures: patient and physician global assessments, pain, disability, and an acute-phase CRP. The ACR50 analysis was implemented in the same way as described above for the ACR20 analysis.

Subjects receiving tildrakizumab (100 mg q12 weeks or 200 mg [q4 and q12 weeks] dose groups) during Part 1 who fail to show clinical response to treatment at Week 24 will discontinue the study drug and enter the washout period per protocol. Week 24 assessments for subjects who discontinue the study drug are recorded at Week 52/EOT. These assessments will be reported as part of the Week 24 visit. Two-sided 95% CI and p-value are based on the CMH test with prior anti-TNF use and Baseline weight as stratification factors. If Mantel-Fleiss criterion is less than 5, pairwise comparisons will be based on Fisher's exact tests after collapsing across levels of the stratification factors. This is noted with a “*” for p-value. Baseline is defined as the last available value before the first dose of study drug.

TABLE 5
CMH Analysis of ACR50 Response Rates up to Week 24 (Missing Response = Non response) -Full Analysis Set

| | | | | | | Comparison to Placebo | | | | |
|---|---|---|---|---|---|---|---|---|---|---|
| | | | | | | | | 95% Confidence | | |
| | | | | | | | | Interval | | |
| | | | | Response | SE | Difference | SE of | Lower | Upper | |
| Visit | Treatment Group | N | n | Rate(%) | (%) | (%) | Difference(%) | (%) | (%) | p-value |
| Week 1 | 200 mg tildrakizumab | 78 | 0 | 0 | 0.00 | | | | | |
| | q4wk | | | | | | | | | |
| | 200 mg tildrakizumab | 79 | 2 | 2.53 | 1.77 | | | | | |
| | q12wk | | | | | | | | | |
| | 100 mg tildrakizumab | 77 | 1 | 1.30 | 1.29 | | | | | |
| | 20 mg tildrakizumab | 78 | 1 | 1.28 | 1.27 | | | | | |
| | Placebo | 79 | 0 | 0 | 0.00 | | | | | |
| | 200 mg tildrakizumab | | | | | | | | | |
| | q4wk vs Placebo | | | | | | | | | |
| | 200 mg tildrakizumab | | | | | 2.53 | 1.77 | −0.93 | 6.00 | 0.4968* |
| | q12wk vs Placebo | | | | | | | | | |
| | 100 mg tildrakizumab | | | | | 1.30 | 1.29 | −1.23 | 3.83 | 0.4936* |
| | vs Placebo | | | | | | | | | |
| | 20 mg tildrakizumab | | | | | 1.28 | 1.27 | −1.21 | 3.78 | 0.4968* |
| | vs Placebo | | | | | | | | | |
| Week 4 | 200 mg tildrakizumab | 78 | 2 | 2.56 | 1.79 | | | | | |
| | q4wk | | | | | | | | | |
| | 200 mg tildrakizumab | 79 | 4 | 5.06 | 2.47 | | | | | |
| | q12wk | | | | | | | | | |
| | 100 mg tildrakizumab | 77 | 7 | 9.09 | 3.28 | | | | | |
| | 20 mg tildrakizumab | 78 | 3 | 3.85 | 2.18 | | | | | |
| | Placebo | 79 | 2 | 2.53 | 1.77 | | | | | |
| | 200 mg tildrakizumab | | | | | 0.03 | 2.52 | −4.90 | 4.96 | >0.9999 |
| | q4wk vs Placebo | | | | | | | | | * |
| | 200 mg tildrakizumab | | | | | 2.53 | 3.03 | −3.42 | 8.48 | 0.6814* |
| | q12wk vs Placebo | | | | | | | | | |
| | 100 mg tildrakizumab | | | | | 6.56 | 3.72 | −0.74 | 13.86 | 0.0960* |
| | vs Placebo | | | | | | | | | |
| | 20 mg tildrakizumab | | | | | 1.31 | 2.80 | −4.18 | 6.81 | 0.6814* |
| | vs Placebo | | | | | | | | | |
| Week 8 | 200 mg tildrakizumab | 78 | 13 | 16.67 | 4.22 | | | | | |
| | q4wk | | | | | | | | | |
| | 200 mg tildrakizumab | 79 | 6 | 7.59 | 2.98 | | | | | |
| | q12wk | | | | | | | | | |
| | 100 mg tildrakizumab | 77 | 10 | 12.99 | 3.83 | | | | | |
| | 20 mg tildrakizumab | 78 | 11 | 14.10 | 3.94 | | | | | |
| | Placebo | 79 | 6 | 7.59 | 2.98 | | | | | |
| | 200 mg tildrakizumab | | | | | 9.00 | 5.17 | −1.13 | 19.13 | 0.0842 |
| | q4wk vs Placebo | | | | | | | | | |
| | 200 mg tildrakizumab | | | | | −0.20 | 4.16 | −8.36 | 7.96 | 0.9623 |
| | q12wk vs Placebo | | | | | | | | | |
| | 100 mg tildrakizumab | | | | | 5.51 | 4.86 | −4.01 | 15.03 | 0.2594 |
| | vs Placebo | | | | | | | | | |
| | 20 mg tildrakizumab | | | | | 6.64 | 4.93 | −3.02 | 16.31 | 0.1840 |
| | vs Placebo | | | | | | | | | |
| Week 12 | 200 mg tildrakizumab | 78 | 14 | 17.95 | 4.35 | | | | | |
| | q4wk | | | | | | | | | |
| | 200 mg tildrakizumab | 79 | 14 | 17.72 | 4.30 | | | | | |
| | q12wk | | | | | | | | | |
| | 100 mg tildrakizumab | 77 | 16 | 20.78 | 4.62 | | | | | |
| | 20 mg tildrakizumab | 78 | 15 | 19.23 | 4.46 | | | | | |
| | Placebo | 79 | 5 | 6.33 | 2.74 | | | | | |
| | 200 mg tildrakizumab | | | | | 11.32 | 5.08 | 1.36 | 21.28 | 0.0286 |
| | q4wk vs Placebo | | | | | | | | | |
| | 200 mg tildrakizumab | | | | | 11.26 | 5.10 | 1.26 | 21.25 | 0.0307 |
| | q12wk vs Placebo | | | | | | | | | |
| | 100 mg tildrakizumab | | | | | 14.47 | 5.42 | 3.84 | 25.10 | 0.0090 |
| | vs Placebo | | | | | | | | | |
| | 20 mg tildrakizumab | | | | | 12.68 | 5.24 | 2.42 | 22.95 | 0.0179 |
| | vs Placebo | | | | | | | | | |
| Week 16 | 200 mg tildrakizumab | 78 | 24 | 30.77 | 5.23 | | | | | |
| | q4wk | | | | | | | | | |
| | 200 mg tildrakizumab | 79 | 22 | 27.85 | 5.04 | | | | | |
| | q12wk | | | | | | | | | |
| | 100 mg tildrakizumab | 77 | 21 | 27.27 | 5.08 | | | | | |
| | 20 mg tildrakizumab | 78 | 16 | 20.51 | 4.57 | | | | | |
| | Placebo | 79 | 4 | 5.06 | 2.47 | | | | | |
| | 200 mg tildrakizumab | | | | | 25.59 | 5.76 | 14.30 | 36.89 | <.0001 |
| | q4wk vs Placebo | | | | | | | | | |
| | 200 mg tildrakizumab | | | | | 22.80 | 5.62 | 11.78 | 33.83 | 0.0001 |
| | q12wk vs Placebo | | | | | | | | | |
| | 100 mg tildrakizumab | | | | | 22.11 | 5.71 | 10.91 | 33.30 | 0.0002 |
| | vs Placebo | | | | | | | | | |
| | 20 mg tildrakizumab | | | | | 15.27 | 5.27 | 4.94 | 25.60 | 0.0045 |
| | vs Placebo | | | | | | | | | |
| Week 20 | 200 mg tildrakizumab | 78 | 35 | 44.87 | 5.63 | | | | | |
| | q4wk | | | | | | | | | |
| | 200 mg tildrakizumab | 79 | 33 | 41.77 | 5.55 | | | | | |
| | q12wk | | | | | | | | | |
| | 100 mg tildrakizumab | 77 | 22 | 28.57 | 5.15 | | | | | |
| | 20 mg tildrakizumab | 78 | 18 | 23.08 | 4.77 | | | | | |
| | Placebo | 79 | 13 | 16.46 | 4.17 | | | | | |
| | 200 mg tildrakizumab | | | | | 28.00 | 7.05 | 14.19 | 41.82 | 0.0001 |
| | q4wk vs Placebo | | | | | | | | | |
| | 200 mg tildrakizumab | | | | | 25.25 | 7.00 | 11.52 | 38.98 | 0.0006 |
| | q12wk vs Placebo | | | | | | | | | |
| | 100 mg tildrakizumab | | | | | 11.99 | 6.56 | −0.86 | 24.84 | 0.0716 |
| | vs Placebo | | | | | | | | | |
| | 20 mg tildrakizumab | | | | | 6.21 | 6.31 | −6.16 | 18.57 | 0.3290 |
| | vs Placebo | | | | | | | | | |
| Week 24 | 200 mg tildrakizumab | 78 | 41 | 52.56 | 5.65 | | | | | |
| | q4wk | | | | | | | | | |
| | 200 mg tildrakizumab | 79 | 40 | 50.63 | 5.62 | | | | | |
| | q12wk | | | | | | | | | |
| | 100 mg tildrakizumab | 77 | 35 | 45.45 | 5.67 | | | | | |
| | 20 mg tildrakizumab | 78 | 31 | 39.74 | 5.54 | | | | | |
| | Placebo | 79 | 19 | 24.05 | 4.81 | | | | | |
| | 200 mg tildrakizumab | | | | | 28.01 | 7.29 | 13.72 | 42.30 | 0.0002 |
| | q4wk vs Placebo | | | | | | | | | |
| | 200 mg tildrakizumab | | | | | 26.16 | 7.33 | 11.80 | 40.52 | 0.0006 |
| | q12wk vs Placebo | | | | | | | | | |
| | 100 mg tildrakizumab | | | | | 21.18 | 7.35 | 6.77 | 35.59 | 0.0048 |
| | vs Placebo | | | | | | | | | |
| | 20 mg tildrakizumab | | | | | 15.29 | 7.20 | 1.17 | 29.41 | 0.0368 |
| | vs Placebo | | | | | | | | | |

Table 6 illustrates CMH analysis of the ACR70 response rates up to Week 24. ACR70 is calculated as a >=70% improvement from baseline in tender and swollen joint counts and >=70% improvement from baseline in three of the five remaining ACR-core set measures: patient and physician global assessments, pain, disability, and an acute-phase CRP. The ACR70 analysis was implemented in the same way as described above for the ACR20 analysis.

TABLE 6
CMH Analysis of ACR70 Response Rates up to Week 24 (Missing Response = Non-response) - Full Analysis Set

| | | | | | | Comparison to Placebo | | | | |
|---|---|---|---|---|---|---|---|---|---|---|
| | | | | | | | | 95% Confidence | | |
| | | | | | | | | Interval | | |
| | | | | Response | SE | Difference | SE of | Lower | Upper | |
| Visit | Treatment Group | N | n | Rate (%) | (%) | (%) | Difference (%) | (%) | (%) | p-value |
| Week 1 | 200 mg tildrakizumab | 78 | 0 | 0 | 0.00 | | | | | |
| | q4wk | | | | | | | | | |
| | 200 mg tildrakizumab | 79 | 1 | 1.27 | 1.26 | | | | | |
| | q12wk | | | | | | | | | |
| | 100 mg tildrakizumab | 77 | 0 | 0 | 0.00 | | | | | |
| | 20 mg tildrakizumab | 78 | 0 | 0 | 0.00 | | | | | |
| | Placebo | 79 | 0 | 0 | 0.00 | | | | | |
| | 200 mg tildrakizumab | | | | | | | | | |
| | q4wk vs Placebo | | | | | | | | | |
| | 200 mg tildrakizumab | | | | | 1.27 | 1.26 | −1.20 | 3.73 | >0.9999* |
| | q12wk vs Placebo | | | | | | | | | |
| | 100 mg tildrakizumab | | | | | | | | | |
| | vs Placebo | | | | | | | | | |
| | 20 mg tildrakizumab | | | | | | | | | |
| | vs Placebo | | | | | | | | | |
| Week 4 | 200 mg tildrakizumab | 78 | 1 | 1.28 | 1.27 | | | | | |
| | q4wk | | | | | | | | | |
| | 200 mg tildrakizumab | 79 | 1 | 1.27 | 1.26 | | | | | |
| | q12wk | | | | | | | | | |
| | 100 mg tildrakizumab | 77 | 0 | 0 | 0.00 | | | | | |
| | 20 mg tildrakizumab | 78 | 1 | 1.28 | 1.27 | | | | | |
| | Placebo | 79 | 1 | 1.27 | 1.26 | | | | | |
| | 200 mg tildrakizumab | | | | | 0.02 | 1.79 | −3.49 | 3.52 | >0.9999* |
| | q4wk vs Placebo | | | | | | | | | |
| | 200 mg tildrakizumab | | | | | 0.00 | 1.78 | −3.49 | 3.49 | >0.9999* |
| | q12wk vs Placebo | | | | | | | | | |
| | 100 mg tildrakizumab | | | | | −1.27 | 1.26 | −3.73 | 1.20 | >0.9999* |
| | vs Placebo | | | | | | | | | |
| | 20 mg tildrakizumab | | | | | 0.02 | 1.79 | −3.49 | 3.52 | >0.9999* |
| | vs Placebo | | | | | | | | | |
| Week 8 | 200 mg tildrakizumab | 78 | 2 | 2.56 | 1.79 | | | | | |
| | q4wk | | | | | | | | | |
| | 200 mg tildrakizumab | 79 | 3 | 3.80 | 2.15 | | | | | |
| | q12wk | | | | | | | | | |
| | 100 mg tildrakizumab | 77 | 2 | 2.60 | 1.81 | | | | | |
| | 20 mg tildrakizumab | 78 | 2 | 2.56 | 1.79 | | | | | |
| | Placebo | 79 | 3 | 3.80 | 2.15 | | | | | |
| | 200 mg tildrakizumab | | | | | −1.23 | 2.80 | −6.72 | 4.25 | >0.9999* |
| | q4wk vs Placebo | | | | | | | | | |
| | 200 mg tildrakizumab | | | | | 0.00 | 3.04 | −5.96 | 5.96 | >0.9999* |
| | q12wk vs Placebo | | | | | | | | | |
| | 100 mg tildrakizumab | | | | | −1.20 | 2.81 | −6.71 | 4.31 | >0.9999* |
| | vs Placebo | | | | | | | | | |
| | 20 mg tildrakizumab | | | | | −1.23 | 2.80 | −6.72 | 4.25 | >0.9999* |
| | vs Placebo | | | | | | | | | |
| Week 12 | 200 mg tildrakizumab | 78 | 8 | 10.26 | 3.44 | | | | | |
| | q4wk | | | | | | | | | |
| | 200 mg tildrakizumab | 79 | 3 | 3.80 | 2.15 | | | | | |
| | q12wk | | | | | | | | | |
| | 100 mg tildrakizumab | 77 | 5 | 6.49 | 2.81 | | | | | |
| | 20 mg tildrakizumab | 78 | 8 | 10.26 | 3.44 | | | | | |
| | Placebo | 79 | 1 | 1.27 | 1.26 | | | | | |
| | 200 mg tildrakizumab | | | | | 8.99 | 3.66 | 1.82 | 16.16 | 0.0177* |
| | q4wk vs Placebo | | | | | | | | | |
| | 200 mg tildrakizumab | | | | | 2.53 | 2.49 | −2.35 | 7.41 | 0.6202* |
| | q12wk vs Placebo | | | | | | | | | |
| | 100 mg tildrakizumab | | | | | 5.23 | 3.08 | −0.80 | 11.26 | 0.1143* |
| | vs Placebo | | | | | | | | | |
| | 20 mg tildrakizumab | | | | | 8.99 | 3.66 | 1.82 | 16.16 | 0.0177* |
| | vs Placebo | | | | | | | | | |
| Week 16 | 200 mg tildrakizumab | 78 | 11 | 14.10 | 3.94 | | | | | |
| | q4wk | | | | | | | | | |
| | 200 mg tildrakizumab | 79 | 10 | 12.66 | 3.74 | | | | | |
| | q12wk | | | | | | | | | |
| | 100 mg tildrakizumab | 77 | 9 | 11.69 | 3.66 | | | | | |
| | 20 mg tildrakizumab | 78 | 7 | 8.97 | 3.24 | | | | | |
| | Placebo | 79 | 2 | 2.53 | 1.77 | | | | | |
| | 200 mg tildrakizumab | | | | | 11.52 | 4.32 | 3.06 | 19.99 | 0.0088 |
| | q4wk vs Placebo | | | | | | | | | |
| | 200 mg tildrakizumab | | | | | 9.96 | 4.13 | 1.87 | 18.06 | 0.0186 |
| | q12wk vs Placebo | | | | | | | | | |
| | 100 mg tildrakizumab | | | | | 9.16 | 4.12 | 1.09 | 17.23 | 0.0271 |
| | vs Placebo | | | | | | | | | |
| | 20 mg tildrakizumab | | | | | 6.44 | 3.69 | −0.78 | 13.67 | 0.0982* |
| | vs Placebo | | | | | | | | | |
| Week 20 | 200 mg tildrakizumab | 78 | 16 | 20.51 | 4.57 | | | | | |
| | q4wk | | | | | | | | | |
| | 200 mg tildrakizumab | 79 | 21 | 26.58 | 4.97 | | | | | |
| | q12wk | | | | | | | | | |
| | 100 mg tildrakizumab | 77 | 11 | 14.29 | 3.99 | | | | | |
| | 20 mg tildrakizumab | 78 | 10 | 12.82 | 3.79 | | | | | |
| | Placebo | 79 | 3 | 3.80 | 2.15 | | | | | |
| | 200 mg tildrakizumab | | | | | 16.48 | 5.09 | 6.50 | 26.45 | 0.0014 |
| | q4wk vs Placebo | | | | | | | | | |
| | 200 mg tildrakizumab | | | | | 22.90 | 5.43 | 12.26 | 33.54 | <.0001 |
| | q12wk vs Placebo | | | | | | | | | |
| | 100 mg tildrakizumab | | | | | 10.52 | 4.56 | 1.58 | 19.46 | 0.0229 |
| | vs Placebo | | | | | | | | | |
| | 20 mg tildrakizumab | | | | | 8.81 | 4.34 | 0.31 | 17.31 | 0.0464 |
| | vs Placebo | | | | | | | | | |
| Week 24 | 200 mg tildrakizumab | 78 | 22 | 28.21 | 5.10 | | | | | |
| | q4wk | | | | | | | | | |
| | 200 mg tildrakizumab | 79 | 23 | 29.11 | 5.11 | | | | | |
| | q12wk | | | | | | | | | |
| | 100 mg tildrakizumab | 77 | 17 | 22.08 | 4.73 | | | | | |
| | 20 mg tildrakizumab | 78 | 13 | 16.67 | 4.22 | | | | | |
| | Placebo | 79 | 8 | 10.13 | 3.39 | | | | | |
| | 200 mg tildrakizumab | | | | | 17.71 | 6.14 | 5.68 | 29.74 | 0.0045 |
| | q4wk vs Placebo | | | | | | | | | |
| | 200 mg tildrakizumab | | | | | 18.58 | 6.07 | 6.68 | 30.49 | 0.0030 |
| | q12wk vs Placebo | | | | | | | | | |
| | 100 mg tildrakizumab | | | | | 11.82 | 5.70 | 0.64 | 22.99 | 0.0415 |
| | vs Placebo | | | | | | | | | |
| | 20 mg tildrakizumab | | | | | 6.16 | 5.36 | −4.35 | 16.67 | 0.2538 |
| | vs Placebo | | | | | | | | | |

Abbreviations: q = every; wk = week; ACR = American College of Rheumatology; CMH = Cochran-Mantel-Haenszel; CRP = C-reactive protein; SE = Standard Error.
N = number of subjects in full analysis set.
n = number of responders.

### 4. Results up to Week 52

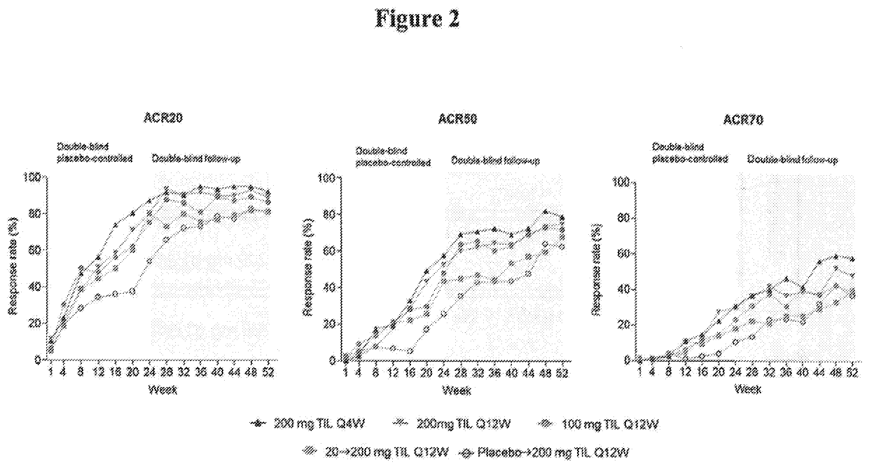

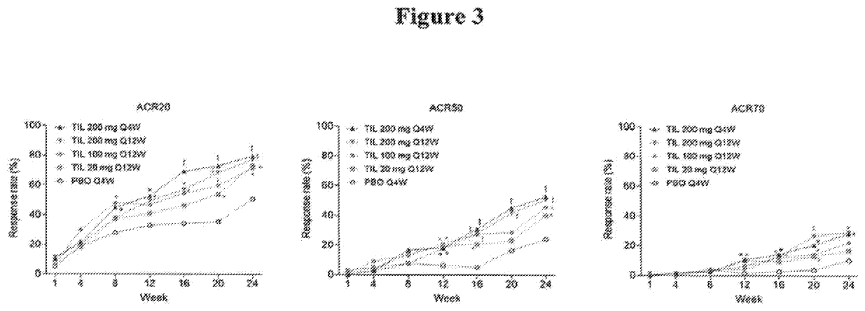

Of 500 patients screened, 391 were randomized and received ≥1 dose of drug. Proportions of ACR20/50/70 responders were superior with tildrakizumab versus placebo through Week 24; after Week 24, responders were further increased for tildrakizumab 20Δ200 mg Q12W and placebo→200 mg Q12W through Week 52 (FIGS. 2 and 3). Other efficacy results are shown in Table 7.

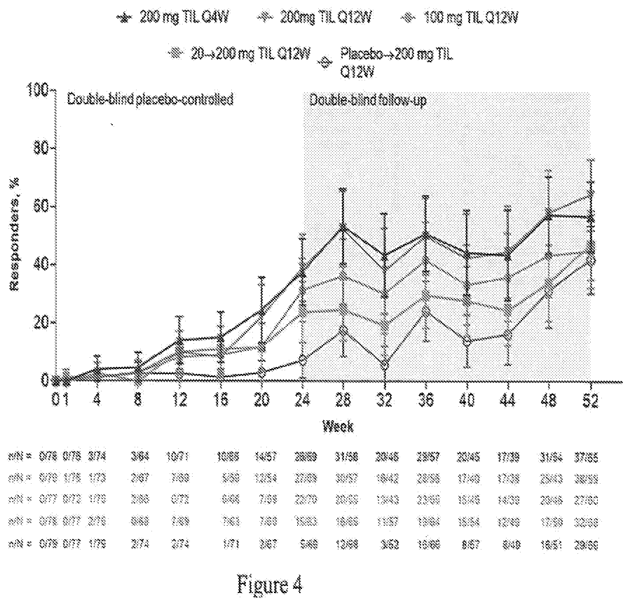

MDA was assessed throughout the study and an MDA response was achieved when 5 of 7 criteria were met. Baseline disease characteristics related to MDA varied little between study arms (Table 8). By Week 24, MDA state was achieved in significantly more patients receiving tildrakizumab versus placebo (0% to 24%-39% versus 0% to 7%; p<0.02 for all groups); the proportion further increased with continued tildrakizumab treatment to Week 52 (45%-64%), including those patients who switched from placebo to tildrakizumab (47%) (FIG. 4).

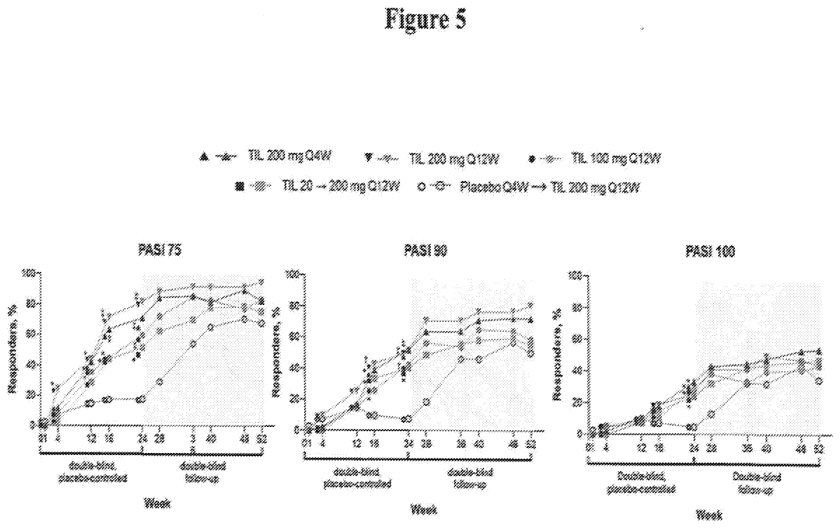

Tildrakizumab treatment significantly increased the proportion of PASI 75/90/100 responders versus placebo at Week 24; the proportion continued to increase thereafter and remained stable through Week 52 (FIG. 5). Similarly, in patients switching from placeboΔtildrakizumab 200 mg Q12W or escalating from tildrakizumab 20Δ200 mg Q12W after Week 24, PASI 75/90/100 response rates increased through Week 36 and remained stable through Week 52. Improvements in skin responses were significant versus placebo as early as Week 4 for PASI 75 in for tildrakizumab 200 mg Q12W. Tildrakizumab had an acceptable safety profile through Week 52.

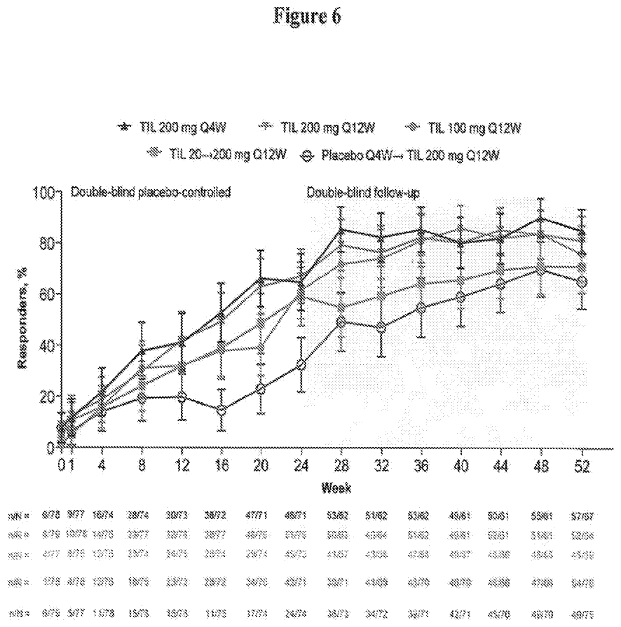

DAS28-CRP was shown to be reliable in PsA and patients achieving scores <3.2 were considered responders. At baseline, disease characteristics were consistent across treatment arms, and 1.3%-7.7% patients had DAS28-CRP scores <3.2 (Table 9). At Week 24, DAS28-CRP response rates increased across all tildrakizumab treatment arms relative to placebo (FIG. 6). After Week 24, response rates continued to increase and were sustained through Week 52, including in patients who switched from placebo to tildrakizumab.

Overall from baseline→Week 24/Week 25→Week 52, 50.4%/39.9% and 2.3%/1.0% of patients experienced a TEAE and serious AE, respectively. The most frequent TEAEs were nasopharyngitis (pooled tildrakizumab arms 5.4%/4.2% vs placebo 6.3%/3.8%) and upper respiratory tract infection (pooled tildrakizumab arms 3.8%/4.2% vs placeob 1.3%/0.0%). One patient (0.3%) discontinued before 24 weeks due to hypertension. From baselineΔWeek 24, 1 case of pyelonephritis and urinary tract infection was reported in the TIL 100 mg Q12W arm and 1 case of chronic tonsillitis was reported in the TIL 20 mg→200 mg Q12W arm. During Week 25→Week 52, 1 malignancy was reported in the TIL 20 mg→200 mg Q12W arm. There were no deaths or major adverse cardiac events.

TABLE 7
Clinical efficacy at Week 52 (W52)

| | | | | TIL | PBO → |
|---|---|---|---|---|---|
| | TIL | TIL | TIL | 20 mg → | TIL |
| | 200 mg | 200 mg | 100 mg | 200 mg | 200 mg |
| | Q4W | Q12W | Q12W | Q12W | Q12W |
| | n = 78 | n = 79 | n = 77 | n = 78 | n = 79 |
| HAQ-DI, BL^{a} | 1.0 | 1.0 | 1.0 | 1.1 | 1.2 |
| W52^{b} | −0.5 | −0.5 | −0.5 | −0.5 | −0.5 |
| LEI, BL^{a} | 1.9 | 1.5 | 2.2 | 2.2 | 1.5 |
| W52^{b} | −1.3 | −1.0 | −1.7 | −1.2 | −1.2 |
| LDI, BL^{a} | 15.0 | 20.8 | 38.7 | 26.1 | 45.4 |
| W52^{b} | −14.5 | −18.9 | −27.1 | −26.2 | −50.4 |
| DAS28-CRP <3.2, | 7.7 | 7.6 | 5.2 | 1.3 | 7.6 |
| %, BL | | | | | |
| %, W52 | 85.1 | 81.3 | 76.3 | 71.1 | 65.3 |
| Minimal disease | 56.9 | 64.4 | 45.0 | 47.1 | 42.0 |
| activity, %, W52 | | | | | |
| PASI 100, %, W52 | 54.0 | 44.4 | 43.9 | 47.5 | 35.0 |
| PASI 90, %, W52 | 72.0 | 80.6 | 58.5 | 55.0 | 50.0 |
| PASI 75, %, W52 | 82.0 | 94.4 | 82.9 | 75.0 | 67.5 |

^{a}Mean at baseline.
^{b}Mean change from baseline.

TABLE 8
Baseline disease characteristics related to minimal disease
activity at Week 52

| | TIL | TIL | TIL | TIL 20 → | PBO → TIL |
|---|---|---|---|---|---|
| | 200 mg | 200 mg | 100 mg | 200 mg | 200 mg |
| | Q4W | Q12W | Q12W | Q12W | Q12W |
| | n = 78 | n = 79 | n = 77 | n = 78 | n = 79 |
| Swollen joint count | 10.4 | 10.0 | 11.0 | 9.4 | 11.8 |
| Tender joint count | 16.6 | 19.5 | 21.3 | 19.0 | 19.7 |
| Patient GADA score | 57.8 | 61.1 | 60.3 | 61.9 | 65.2 |
| Patient pain | 55.4 | 59.6 | 59.2 | 60.9 | 64.2 |
| assessment | | | | | |
| Enthesitis (LEI) | 1.9 | 1.5 | 2.2 | 2.2 | 1.5 |
| score* | | | | | |
| PASI^{†} | 7.6 | 6.2 | 8.8 | 6.6 | 5.0 |
| HAQ-DI score | 1.0 | 1.0 | 1.0 | 1.1 | 1.2 |

Data are reported as mean.
*Total patients analysed (n) = 76, 79, 76, 78, 78, respectively.
^{†}Total patients analysed (n) = 75, 79, 76, 75, 75, respectively.

TABLE 9
Baseline disease characteristics related to DAS28-CRP

| | TIL | TIL | TIL | TIL 20 → | PBO → TIL |
|---|---|---|---|---|---|
| | 200 mg | 200 mg | 100 mg | 200 mg | 200 mg |
| | Q4W | Q12W | Q12W | Q12W | Q12W |
| | n = 78 | n = 79 | n = 77 | n = 78 | n = 79 |
| Baseline DAS28- | 6 (7.7) | 6 (7.6) | 4 (5.2) | 1(1.3) | 6 (7.6) |
| CRP <3.2, n (%) | | | | | |
| hsCRP, mg/L | 7.8 ± 18.6 | 10.5 ± 14.4 | 10.6 ± 20.0 | 10.7 ± 14.0 | 13.0 ± 20.8 |
| ESR, mm/h* | 22.8 ± 18.9 | 22.5 ± 19.8 | 24.7 ± 19.8 | 27.2 ± 20.7 | 26.9 ± 20.5 |
| Swollen joint count (66) | 10.4 ± 7.4 | 10.0 ± 8.0 | 11.0 ± 8.2 | 9.4 ± 6.4 | 11.8 ± 9.8 |
| Tender joint count (68) | 16.6 ± 11.9 | 19.5 ± 13.9 | 21.3 ± 14.8 | 19.0 ± 13.0 | 19.7 ± 14.7 |
| PtGA | 57.8 ± 18.3 | 61.1 ± 20.7 | 60.3 ± 20.2 | 61.9 ± 17.4 | 65.2 ± 18.1 |

Data are reported as mean ± standard deviation unless otherwise stated.
*Total pts analysed (n) = 71, 69, 70, 68, 62, respectively.
ESR, erythrocyte sedimentation rate; hsCRP, high-sensitivity C-Reactive Protein; PBO, placebo; PtGA, Patient Global Assessment; pts, patients; Q4W, every 4 weeks; Q12W, every 12 weeks; TIL, tildrakizumab.

### 5. Conclusion

Tildrakizumab demonstrated a surprisingly high efficacy in this clinical trial with a Q12Wk dosing regimen. Tildrakizumab was found to be significantly more efficacious than

Placebo in treatment of joint manifestations of active Psoriatic Arthritis as measured by ACR20, ACR50, and ACR70 response criteria. While the disclosure has been described in terms of various embodiments, it is understood that variations and modifications will occur to those skilled in the art. Therefore, it is intended that the appended claims cover all such equivalent variations that come within the scope of the disclosure as claimed. In addition, the section headings used herein are for organizational purposes only and are not to be construed as limiting the subject matter described.

Each embodiment described herein may be combined with any other embodiment or embodiments unless clearly indicated to the contrary. In particular, any feature or embodiment indicated as being preferred or advantageous may be combined with any other feature or features or embodiment or embodiments indicated as being preferred or advantageous, unless clearly indicated to the contrary.

What is claimed is:

1. A method of treating psoriatic arthritis comprising administering an anti-IL-23p19 antibody hum 13B8-b to a patient in need thereof, wherein the patient is subcutaneously administered a first dose of the antibody on week 0 and a subsequent dose at every 12 weeks thereafter; and wherein the antibody hum13B8-b comprises:
(i) a light chain polypeptide comprising the amino acid sequence of SEQ ID NO: 1; and
(ii) a heavy chain polypeptide comprising the amino acid sequence of SEQ ID NO: 2.

2. The method according to claim 1, wherein the first dose and the subsequent dose are the same.

3. The method according to claim 1, wherein the first dose and the subsequent dose are different.

4. The method according to claim 1, wherein the first dose is 100 mg.

5. The method according to claim 1, wherein the first dose is 200 mg.

6. The method according to claim 1, wherein the subsequent dose is 100 mg.

7. The method according to claim 1, wherein the subsequent dose is 200 mg.

8. The method according to claim 2, wherein the first dose and the subsequent dose are 100 mg.

9. The method according to claim 2, wherein the first dose and the subsequent dose are 200 mg.

10. The method according to claim 3, wherein the first dose is 100 mg and the subsequent dose is 200 mg.

11. The method according to claim 3, wherein the first dose is 200 mg and the subsequent dose is 100 mg.

12. The method according to claim 1, wherein the treatment comprises administration of the subsequent dose at every 12 weeks at least up to 24 weeks.

13. The method according to claim 1, wherein the treatment comprises administration of the subsequent dose at every 12 weeks at least up to 36 weeks.

14. The method according to claim 1, wherein the treatment comprises administration of the subsequent dose at every 12 weeks at least up to 48 weeks.

15. The method according to claim 1, wherein the treatment comprises administration of the subsequent dose at every 12 weeks at least up to 60 weeks.

16. The method according to claim 1, wherein the treatment comprises administration of the subsequent dose at every 12 weeks at least up to 72 weeks.

17. A method of treating psoriatic arthritis comprising administering to a patient in need thereof a therapeutically effective amount of an anti-IL-23p19 antibody hum13B8-b, wherein the treatment results in at least 20% improvement from baseline value of tender joint count and swollen joint count; and wherein the antibody hum13B8-b comprises:
(i) a light chain polypeptide comprising the amino acid sequence of SEQ ID NO: 1; and
(ii) a heavy chain polypeptide comprising the amino acid sequence of SEQ ID NO: 2.

18. The method according to claim 17, wherein the treatment further results in at least 20% improvement from baseline for at least three of the five parameters selected from the group consisting of (i) Physician Global Assessment of disease activity, (ii) Patient Global Assessment of disease activity, (iii) Patient pain assessment, (iv) patient self-assessed disability, and (v) acute-phase CRP.

19. A method of treating psoriatic arthritis comprising administering to a patient in need thereof a therapeutically effective amount of an anti-IL-23p19 antibody hum13B8-b, wherein the treatment results in at least 50% improvement from baseline value of tender joint count and swollen joint count; and wherein the antibody hum13B8-b comprises:
(i) a light chain polypeptide comprising the amino acid sequence of SEQ ID NO: 1; and
(ii) a heavy chain polypeptide comprising the amino acid sequence of SEQ ID NO: 2.

20. The method according to claim 19, wherein the treatment further results in at least 50% improvement from baseline for at least three of the five parameters selected from the group consisting of (i) Physician Global Assessment of disease activity, (ii) Patient Global Assessment of disease activity, (iii) Patient pain assessment, (iv) patient self-assessed disability, and (v) acute-phase CRP.

21. A method of treating psoriatic arthritis comprising administering to a patient in need thereof a therapeutically effective amount of an anti-IL-23p19 antibody hum13B8-b, wherein the treatment results in at least 70% improvement from baseline value of tender joint count and swollen joint count; and wherein the antibody hum13B8-b comprises:
(i) a light chain polypeptide comprising the amino acid sequence of SEQ ID NO: 1; and
(ii) a heavy chain polypeptide comprising the amino acid sequence of SEQ ID NO: 2.

22. The method according to claim 21, wherein the treatment further results in at least 70% improvement from baseline for at least three of the five parameters selected from the group consisting of (i) Physician Global Assessment of disease activity, (ii) Patient Global Assessment of disease activity, (iii) Patient pain assessment, (iv) patient self-assessed disability, and (v) acute-phase CRP.

23. A method of determining the efficacy of an anti-IL-23p19 antibody for the treatment of psoriatic arthritis comprising administering an anti-IL-23p19 antibody hum13B8-b to a patient, wherein the patient is subcutaneously administered a first dose of the antibody on week 0 and a subsequent dose at every 12 weeks after the first dose; and wherein the antibody hum13B8-b comprises:
(i) a light chain polypeptide comprising the amino acid sequence of SEQ ID NO: 1; and
(ii) a heavy chain polypeptide comprising the amino acid sequence of SEQ ID NO: 2;
and wherein an ACR20 response value of at least about 40% at week 24 or week 52 indicates the efficacy of the antibody.

24. The method according to claim 23, wherein an ACR20 response value of at least about 50% at week 24 or week 52 indicates the efficacy of the antibody.

25. The method according to claim 23, wherein an ACR20 response value of at least about 60% at week 24 or week 52 indicates the efficacy of the antibody.

26. A method of determining the efficacy of an anti-IL-23p19 antibody for the treatment of psoriatic arthritis comprising administering an anti-IL-23p19 antibody hum13B8-b to a patient, wherein the patient is subcutaneously administered a first dose of the antibody on week 0 and a subsequent dose at every 12 weeks after the first dose; and wherein the antibody hum13B8-b comprises:
(i) a light chain polypeptide comprising the amino acid sequence of SEQ ID NO: 1; and
(ii) a heavy chain polypeptide comprising the amino acid sequence of SEQ ID NO: 2; and wherein an ACR50 response value of at least about 20% at week 24 or week 52 indicates the efficacy of the antibody.

27. The method according to claim 26, wherein an ACR50 response value of at least about 25% at week 24 or week 52 indicates the efficacy of the antibody.

28. The method according to claim 26, wherein an ACR50 response value of at least about 30% at week 24 or week 52 indicates the efficacy of the antibody.

29. A method of determining the efficacy of an anti-IL-23p19 antibody for the treatment of psoriatic arthritis comprising administering an anti-IL-23p19 antibody hum13B8-b to a patient, wherein the patient is subcutaneously administered a first dose of the antibody on week 0 and a subsequent dose at every 12 weeks after the first dose; and wherein the antibody hum13B8-b comprises:
(i) a light chain polypeptide comprising the amino acid sequence of SEQ ID NO: 1; and
(ii) a heavy chain polypeptide comprising the amino acid sequence of SEQ ID NO: 2;
and wherein an ACR70 response value of at least 10% at week 24 or week 52 indicates the efficacy of the antibody.

30. The method according to claim 29, wherein an ACR50 response value of at least about 12% at week 24 or week 52 indicates the efficacy of the antibody.

31. The method according to claim 29, wherein an ACR50 response value of at least about 15% at week 24 or week 52 indicates the efficacy of the antibody.

32. The method according to any one of claims 23-31, wherein the first dose and the subsequent dose are same.

33. The method according to any one of claims 23-31, wherein the first dose and the subsequent dose are different.

34. The method according to any one of claims 23-31, wherein the first dose is 100 mg.

35. The method according to any one of claims 23-31, wherein the first dose is 200 mg.

36. The method according to any one of claims 23-31, wherein the subsequent dose is 100 mg.

37. The method according to any one of claims 23-31, wherein the subsequent dose is 200 mg.

38. The method according to any one of claims 23-31, wherein the first dose and the subsequent dose are 100 mg.

39. The method according to any one of claims 23-31, wherein the first dose and the subsequent dose are 200 mg.

40. A method of determining the efficacy of an anti-IL-23p19 antibody for the treatment of psoriatic arthritis comprising administering an anti-IL-23p19 antibody hum13B8-b to a patient, wherein the patient is subcutaneously administered a first dose of the antibody on week 0 and a subsequent dose at every 12 weeks thereafter; and wherein the antibody hum13B8-b comprises:
(i) a light chain polypeptide comprising the amino acid sequence of SEQ ID NO: 1; and (ii) a heavy chain polypeptide comprising the amino acid sequence of SEQ ID NO: 2;
and wherein at least a 75% improvement from baseline value in Psoriasis Area and Severity Index at week 52 indicates the efficacy of the antibody.

41. The method according to claim 40, wherein at least 90% improvement from baseline value in Psoriasis Area and Severity Index at week 52 indicates the efficacy of the antibody.

42. The method according to claim 40, wherein at least 100% improvement from baseline value in Psoriasis Area and Severity Index at week 52 indicates the efficacy of the antibody.

43. A method of determining the efficacy of an anti-IL-23p19 antibody for the treatment of psoriatic arthritis comprising administering an anti-IL-23p19 antibody hum13B8-b to a patient, wherein the patient is subcutaneously administered a first dose of the antibody on week 0 and a subsequent dose at every 12 weeks thereafter; and wherein the antibody hum13B8-b comprises:
(i) a light chain polypeptide comprising the amino acid sequence of SEQ ID NO: 1; and (ii) a heavy chain polypeptide comprising the amino acid sequence of SEQ ID NO: 2; and
wherein a reduced DAS28-CRP score from baseline value at week 52 indicates the efficacy of the antibody.

44. A method of determining the efficacy of an anti-IL-23p19 antibody for the treatment of psoriatic arthritis comprising administering an anti-IL-23p19 antibody hum13B8-b to a patient, wherein the patient is subcutaneously administered a first dose of the antibody on week 0 and a subsequent dose at every 12 weeks thereafter; and wherein the antibody hum13B8-b comprises:
(i) a light chain polypeptide comprising the amino acid sequence of SEQ ID NO: 1; and (ii) a heavy chain polypeptide comprising the amino acid sequence of SEQ ID NO: 2; and
wherein a statistically significant improvement in disease activity as determined by the minimal disease activity (MDA) criteria at week 52 indicates the efficacy of the antibody.


SEQUENCE LISTING (8 sequences)

SEQ ID NO: 1 - protein, 214 aa, Artificial Sequence
  hum13B8-b light chain
DIQMTQSPSSLSASVGDRVTITCRTSENIYSYLAWYQQKPGKAPKLLIYNAKTLAEGVPSRFSGSGSGTDFTLTISSLQPEDFATYYCQHHYGIPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 446 aa, Artificial Sequence
  hum13B8-b heavy chain
QVQLVQSGAEVKKPGASVKVSCKASGYIFITYWMTWVRQAPGQGLEWMGQIFPASGSADYNEKFEGRVTMTTDTSTSTAYMELRSLRSDDTAVYYCARGGGGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 10 aa, Artificial Sequence
  HC CDR1
GYIFITYWMT

SEQ ID NO: 4 - protein, 17 aa, Artificial Sequence
  HC CDR2
QIFPASGSADYNEKFEG

SEQ ID NO: 5 - protein, 7 aa, Artificial Sequence
  HC CDR3
GGGGFAY

SEQ ID NO: 6 - protein, 11 aa, Artificial Sequence
  LC CDR1
RTSENIYSYLA

SEQ ID NO: 7 - protein, 7 aa, Artificial Sequence
  LC CDR2
NAKTLAE

SEQ ID NO: 8 - protein, 9 aa, Artificial Sequence
  LC CDR3
QHHYGIPFT